SEQUENCE LISTING (13 sequences)

SEQ ID NO: 1 - DNA, 21 bp, artificial sequence
  VEFG forward primer
ccatgaactttctgctgtctt

SEQ ID NO: 2 - DNA, 20 bp, artificial sequence
  VEGF Reverse primer
atcgcatcaggggcacacag

SEQ ID NO: 3 - DNA, 20 bp, artificial sequence
  GAPDH forward primer
aatcccatcaccatcttcca

SEQ ID NO: 4 - DNA, 20 bp, artificial sequence
  GAPDH reverse primer
gtcatcatatttggcaggtt

SEQ ID NO: 5 - DNA, 20 bp, artificial sequence
  VEGFR1 forward primer
tgggacagtagaaagggctt

SEQ ID NO: 6 - DNA, 21 bp, artificial sequence
  VEGFR1 reverse primer
ggtccactccttacacgacaa

SEQ ID NO: 7 - DNA, 20 bp, artificial sequence
  GAPDH forward primer
aatcccatcaccatcttcca

SEQ ID NO: 8 - DNA, 20 bp, artificial sequence
  GAPDH reverse primer
gtcatcatatttggcaggtt

SEQ ID NO: 9 - DNA, 32 bp, artificial sequence
  pGal4-Sp3B sense primer
tccgtcgacgcaacagcgtttctgcagctacc

SEQ ID NO: 10 - DNA, 33 bp, artificial sequence
  pGal4-Sp3B anti-sense primer
tatctagaatcagccttgaattgggtgcacctg

SEQ ID NO: 11 - DNA, 32 bp, artificial sequence
  pGal4-Sp3CD sense primer
tccggatccgcctgccgttggctatagcaaat

SEQ ID NO: 12 - DNA, 35 bp, artificial sequence
  pGal4-Sp3CD anti-sense primer
gtatgtcgacatcagaagccattgccactgatatt

SEQ ID NO: 13 - DNA, 32 bp, artificial sequence; identical to SEQ ID NO: 11
  pGal4-Sp3D sense primer

(19) United States
(12) Patent Application Publication
(10) Pub. No.: US 2008/0261911 A1
(43) Pub. Date: Oct. 23, 2008

(54) Uses of diphenyl/diphenylamine carboxylic acids

(76) Inventors: Stephen H. Safe, College Station, TX (US); Maen Abdelrahim, Orlando, FL (US)

Correspondence Address: Benjamin Aaron Adler;ADLER & ASSOCIATES, 8011 Candle Lane, Houston, TX, 77071, US

(21) Appl. No.: 12/069,529

(22) Filed: Feb. 11, 2008

Related U.S. Application Data
(63) Continuation-in-part of application No. 11/728,566, filed on Mar. 26, 2007.
(60) Provisional application No. 60/785,730, filed on Mar. 24, 2006.

(51) Int. Cl. A61K 31/192; C12N 5/00; A61K 31/195; A61P 35/00; A61K 31/7105

(52) U.S. Cl. 514/44; 435/375; 514/567; 514/568

(57) ABSTRACT

The present invention demonstrates that chemical-induced degradation of Sp proteins by a specific sub-class of non-steroidal anti-inflammatory drugs inhibited cancer cell growth, angiogenesis and metastasis of cancer cells. The inhibitory effects of these compounds were demonstrated in vitro and in vivo. Hence, the results discussed herein indicate that these compounds can be used to inhibit cell growth, angiogenesis, and metastasis in cancers such as pancreatic cancer, esophageal cancer, breast cancer, prostate cancer, colon cancer, bladder cancer, and ovarian cancer.

### CROSS-REFERENCE TO RELATED APPLICATION

This is a continuation-in-part of U.S. Ser. No. 11/728,566, filed Mar. 26, 2007, which claims benefit of provisional patent application 60/785,730, filed Mar. 24, 2006, now abandoned.

### BACKGROUND OF THE INVENTION

1. Field of the Invention

The present invention relates to the fields of cell signaling pertaining to tumor cell growth, angiogenesis and metastasis. More specifically, the present invention discloses degradation of Sp family proteins by a specific sub-class of non-steroidal anti-inflammatory drugs (NSAIDs) and related compounds, which results in inhibition of growth, angiogenesis and metastasis of pancreatic cancer.

2. Description of the Related Art

Development of novel therapies for treating pancreatic cancer and other highly aggressive tumors requires a basic understanding of their critical growth regulatory and angiogenic pathways. Pancreatic carcinoma is the fourth leading cause of cancer mortality in the US, with more than 28,000 deaths attributed to this disease each year. Pancreatic cancer is associated with a death:incidence ratio of approximately 0.99. The incidence of pancreatic cancer in the US has increased nearly three-fold from 1920 to 1978. Pancreatic cancer is characterized by a high metastatic potential and rapid progression with a median survival rate of only 24 weeks in untreated cases. Due to local invasion and/or metastasis, only 15-20% of pancreatic cancer patients qualify for surgical intervention. For locally advanced, unresectable, and metastatic disease, treatment is palliative at best and usually consists of 5-fluorouracil or gemcitabine alone, or in combination with radiotherapy. Unfortunately, despite the moderate success of gemcitabine (2′, 2′-difluorodeoxycitidine) median survival rates remain under 6 months for patients with metastatic disease. Given the poor performance of existing therapies, there is an increasing need to develop alternative drugs that target specific pathways that inhibit angiogenesis and tumor growth/regression.

Transcription factors are now recognized as targets for development of new anti-cancer drugs, and Sp-dependent gene expression is known to play critical roles in tumor development, growth and metastasis. Sp1 is over expressed in pancreatic cancer compared to normal tissues and several studies have linked elevated Sp protein expression to up regulation of genes that are involved in pancreatic tumor growth and metastasis and these include p27 (suppressor) and vascular endothelial growth factor (VEGF) and its receptors. Sp proteins play a critical role in growth and metastasis of cancer (Bouwman et al, 2002; Black et al, 1 2001; Safe et al, 2004), and there is evidence that Sp1 expression is a negative prognostic factor for survival in some cancer patients. These observations are not surprising since Sp1 and other Sp proteins that bind GC-rich promoter sites are transcription factors that regulate key sets of genes responsible for cancer cell proliferation and angiogenesis.

Previous studies showed Sp1 protein interactions with a proximal GC-rich motif in the vascular endothelial growth factor (VEGF) promoter was important for vascular endothelial growth factor expression (Shi et al, 2001), and RNA interference was used to determine the role of Sp1, Sp3 and Sp4 in mediating expression of this important angiogenic factor (Abdelrahim et al, 2004). Using a series of constructs containing vascular endothelial growth factor promoter inserts, it was initially shown that Sp1 and Sp3 were required for transactivation, and this was primarily dependent on proximal GC-rich motifs. Previous, studies have demonstrated that Sp4 was expressed in Panc-1 cells, and RNA interference assays suggested that Sp4 cooperatively interacted with Sp1 and Sp3 to activate vascular endothelial growth factor promoter constructs in these cells. However, the relative contributions of Sp proteins to vascular endothelial growth factor expression were variable among different pancreatic cancer cell lines. Small inhibitory RNAs for Sp3, but not Sp1 or Sp4, inhibited phosphorylation of retinoblastoma protein, blocked G₀/G₁ to S-phase progression, and up regulated p27 protein/promoter activity of Panc-1 cells. Similar results were observed in other pancreatic cancer cells, suggesting that Sp3-dependent growth of pancreatic cancer cells is caused by inhibition of p27 expression (Abdelrahim et al, 2004). These data clearly demonstrate a critical role for Sp proteins for growth and angiogenesis of pancreatic cancer cells, and targeted degradation of these proteins would be highly advantageous for treatment of pancreatic cancer.

Non-steroidal anti-inflammatory drugs comprise large chemically heterogeneous groups of compounds, which suppress inflammation by non-selectively inhibiting activity of cyclooxygenase-1 (COX-1) and cyclooxygenase-2 (COX-2) isoforms. Non-steroidal anti-inflammatory (NSAID) drugs are classified as belonging to one of the carboxylic acid groups, which includes diphenyl/diphenyl amine carboxylic acids, to one of the enolic acid groups or is classified as a coxib or as a gold salt. Generally, non-steroidal anti-inflammatory drugs alleviate pain and fever, therefore, are used widely for the treatment of inflammatory disorders and conditions, such as rheumatoid arthritis, gout, bursitis, painful menstruation, and headache.

Studies have indicated that regularly taking aspirin or indomethacin, a carboxylic acid indole analog, provides a 40-50% reduction in relative risk of death by colon cancer (Shi et al., 2001). U.S. Pat. Nos. 6,207,700 and 6,399,647 describe a method of treating animals having cancer by administration of secondary amide derivatives of indomethacin. U.S. Pat. No. 5,914,322 discloses topical formulations of hyaluronic acid and a non-steroidal anti-inflammatory drug, such as diclofenac, indomethacin, naproxen, a trimethamine salt of ketorolac, ibuprofen, piroxicam, propionic acid derivatives, acetylsalicylic acid, and flunixin are useful in treating primary and metastatic skin cancers and other skin disorders.

The role of non-steroidal anti-inflammatory drugs in both prevention and treatment of colon cancer has been extensively investigated. Celecoxib, a coxib non-steroidal anti-inflammatory drug, has demonstrated anti-angiogenic and anti-tumor activity against colon cancer (Wei et al, 2004). Hence, there is evidence from epidemiology studies that non-steroidal anti-inflammatory drugs, such as aspirin and some cyclooxygenase-2 inhibitors, decreased the incidence and/or mortality of colon cancer. Patients with familial adenomatous polyposis coli are highly susceptible for development of colon cancer and these individuals have been successfully treated with the cyclooxygenase-2 inhibitor, sulindac. Laboratory animal and cell culture studies also confirm the efficacy of non-steroidal anti-inflammatory drugs for inhibiting growth of colon cancer and tumors derived from other tissues.

It is also apparent that the anti-cancer activities of non-steroidal anti-inflammatory drugs and cyclooxygenase-2 inhibitors can be both cyclooxygenase-2-dependent and -independent. Epidemiological studies on the association of non-steroidal anti-inflammatory drugs with decreased risk/lower incidence of other cancers have been reported; however, the linkages are more variable and somewhat inconsistent (Thun et al, 2002; Thun et al, 1993; Egan et al, 1996, Harris et al, 1999; Sharp et al, 2000; Khuder et al, 2001; Norrish et al, 1998; Cramer et al, 1998; Rodriguez et al, 1998; Tavani et al, 2000; Jacobs et al, 2005a; Jacobs et al, 2005b; Lindblad et al, 2005). For example, several cohort studies report that breast cancer incidence is decreased with increasing aspirin/non-steroidal anti-inflammatory drugs use in some cohorts but other studies indicate that aspirin and other non-steroidal anti-inflammatory drugs may only provide minimal protection against breast cancer. A recent large cohort study concluded, “that long duration regular non-steroidal anti-inflammatory drugs use is associated with modestly reduced risk of prostate cancer” (Jacobs et al, 2005a). Limited studies on pancreatic cancer suggest that decreased incidence of this disease was not correlated with aspirin/non-steroidal anti-inflammatory drugs use (Schernhammer et al, 2004; Jacobs et al, 2004; Anderson et al, 2002; Menezes et al, 2002).

Non-steroidal anti-inflammatory drugs/cyclooxygenase-2 inhibitors modulate several pathways in cancer cell lines that lead to inhibition of growth, apoptosis and anti-angiogenesis, and cyclooxygenase-2 inhibitors are being investigated for colon cancer prevention and chemotherapy (Thun et al, 2002). Although prolonged use of non-steroidal anti-inflammatory drugs may decrease incidence of some human cancers (chemoprevention), non-steroidal anti-inflammatory drugs also exhibit anti-tumor activities in models for several cancers. For example, laboratory animal studies show that non-steroidal anti-inflammatory drugs/cyclooxygenase-2 inhibitors such as aspirin, indomethacin, sulindac and celecoxib suppress carcinogen-induced or xenograft orthotopic models of lung, mast cell, fibrosarcoma, esophageal, bladder, pancreatic and mammary cancers (Khuder et al, 2001; Norrish et al, 1998; Cramer et al, 1998; Rodriguez et al, 1998; Tavani et al, 2000; Jacobs et al, 2005a; Jacobs et al, 2005b; Lindblad et al, 2005; Schernhammer et al, 2004; Jacobs et al, 2004). Although the mechanisms of these anti-tumorigenic effects induced by non-steroidal anti-inflammatory drugs are not completely understood, there is strong evidence that non-steroidal anti-inflammatory drugs inhibit cancer cell growth through modulation of cell cycle genes. Moreover, in combination with these anti-proliferative properties, non-steroidal anti-inflammatory drugs also induce apoptosis and exhibit anti-angiogenic activities (Tarnawski and Jones, 2003; Taketo, 1998a; Taketo, 1998b).

Wei and coworkers (Bouwman et al, 2002) first reported that celecoxib decreased cell/tumor growth, Sp1 and vascular endothelial growth factor expression in pancreatic cancer cells and in tumors from nude mice bearing FG pancreatic cancer cells (orthotopic and xenograft models). A previous study using colon cancer cells as a model showed that celecoxib decreased Sp1 and Sp4 (but not Sp3) protein degradation and this was also accompanied by decreased expression of vascular endothelial growth factor (Baker et al, 2002). Thus, the profile of non-steroidal anti-inflammatory drugs-induced responses in cancer cells/tumors is highly desirable for an anti-cancer drug, for the development of non-steroidal anti-inflammatory drugs (including cyclooxygenase-2 inhibitors) as a new class of mechanism-based drugs for treating pancreatic cancer.

Thus, the prior art is still deficient in cancer therapies employing non-steroidal anti-inflammatory drugs as anti-tumorigenic and anti-angiogenic agents. More specifically, the prior art is deficient in chemotherapy regimens utilizing diphenyl/diphenyl amine carboxylic acids as therapeutic agents to degrade Sp proteins in cancer cells. The present invention fulfills this long-standing need and desire in the art.

### SUMMARY OF THE INVENTION

The present invention is directed to a method of inducing degradation of one or more Sp family of transcription factors. Such a method comprises contacting a cancer cell with a non-steroidal anti-inflammatory drug, thereby inducing degradation of one or more Sp transcription factors.

The present invention is also directed to a method of treating a cancer in an individual. This method comprises administering a pharmacologically effective amount of diphenyl/diphenylamine carboxylic acid to the individual, thereby treating the cancer in the individual.

The present invention is further directed to a method of treating pancreatic cancer in an individual. Such a method comprises administering a pharmacologically effective amount of tolfenamic acid, where the tolfenamic acid inhibits proliferation, angiogenesis and metastasis of the pancreatic cancer, thereby treating the pancreatic cancer in the individual.

The present invention is also directed to a method of treating an esophageal cancer in an individual. Such a method comprises administering a pharmacologically effective amount of tolfenamic acid, where the tolfenamic acid inhibits proliferation, angiogenesis and metastasis of the esophageal cancer, thereby treating the esophageal cancer in the individual.

The present invention is further directed to a method of reducing toxicity of a cancer therapy in an individual in need thereof. Such a method comprises administering to the individual a diphenyl/diphenylamine carboxylic acid and another chemotherapeutic drug, where the dosage of the chemotherapeutic drug administered is lower than the dosage required when said chemotherapeutic drug is administered singly, thereby reducing the toxicity of the cancer therapy in the individual.

The present invention is also directed to a method of treating cancer in an individual, consisting of administering a pharmacologically effective amount of diphenyl/diphenylamine carboxylic acid; and a small interfering RNA (siRNA) specific for one or more Sp transcription factors, to the individual, thereby treating the cancer in the individual.

The present invention is further directed to a method of inhibiting an angiogenic response of a tumor in an individual consisting of administering a pharmacologically effective amount of diphenyl/diphenylamine carboxylic acid; and a siRNA specific for one or more Sp transcription factors, to the individual, thereby inhibiting the angiogenic response of the tumor in said individual.

The present invention is also directed to a method of inhibiting tumor metastasis in an individual consisting of administering a pharmacologically effective amount of diphenyl/diphenylamine carboxylic acid; and a siRNA specific for one or more Sp transcription factors, to the individual, thereby inhibiting tumor metastasis in said individual.

### BRIEF DESCRIPTION OF THE DRAWINGS

FIGS. 1A-1D show the effects of non-steroidal anti-inflammatory drugs on Sp1, Sp3 and Sp4 protein expression in pancreatic cancer cells. FIG. 1A show results of screening of NSAIDs/COX-1/2 inhibitors for Sp protein degradation in Panc-1 cells. Cells were treated with 50 μM non-steroidal anti-inflammatory drugs/COX-1/2 inhibitors for 48 hr and whole cell lysates were analyzed by Western blot analysis. FIG. 1B shows the quantitation of Sp proteins after treatment with non-steroidal anti-inflammatory drugs/COX-1/2 inhibitors. The results in FIG. 1A were determined in duplicate and the relative percentage Sp1, Sp3 and Sp4 levels in selected treated versus control (dimethyl sulfoxide (DMSO); all values set at 100%) groups are presented as the average of two duplicate determinations. Protein band intensities were standardized based on β-tubulin protein as a loading control. Histone deacetylase (HDAC) protein is also shown and was unaltered by the treatments. Effects of select on Sp protein in Panc-1 (FIG. 1C) and L3.6pl cells (FIG. 1D) are also shown. Panc-1 cells were treated with DMSO, 50 μM ampiroxicam or tolfenamic acid for 24 or 48 hr, and Sp1, Sp3 and Sp4 protein levels were determined in whole cell lysates by Western blot analysis. Protein band intensities were normalized to β-tubulin and protein levels are presented as means ±SE for three replicate determinations for each treatment group. Significantly (p<0.05) decreased protein levels are indicated by an asterisk.

FIGS. 2A-2G show decreased transactivation in pancreatic cancer cells transfected with pVEGF1 or pVEGF2 constructs and treated with non-steroidal anti-inflammatory drugs. Transfection of Panc-1 cells with pVEGF1 and pVEGF2 and treatment with 50 μM ampiroxicam and tolfenamic acid (FIGS. 2A, 2B) or diclofenac sodium (FIGS. 2C, 2D) with pVEGF1 or pVEGF2 constructs. Cells were transfected with pVEGF1 or pVEGF2 treated with DMSO, 50 μM ampiroxicam, tolfenamic acid, or diclofenac sodium, and luciferase activity determined. Transfection of L3.6pl cells with pVEGF1 (FIG. 2E) or pVEGF2 constructs (FIG. 2F). Cells were transfected with pVEGF1 or pVEGF2 constructs, treated with DMSO, 50 μM ampiroxicam or tolfenamic acid, and luciferase activity determined. FIG. 2G shows concentration-dependent effects of non-steroidal anti-inflammatory drugs on transactivation. Panc-1 cells were transfected with pVEGF2, treated with 20, 40, 60 or 80 μM ampiroxicam or tolfenamic acid, and luciferase determined. Results presented herein are means ±SE for three separate determinations per treatment group, and significant (p<0.05) decrease in luciferase activity is indicated by an asterisk.

FIGS. 3A-3B show effects of non-steroidal anti-inflammatory drugs on binding of nuclear extracts from Panc-1 cells to VEGF32P. Nuclear extracts from Panc-1 cells treated with DMSO, 50 μM ampiroxicam or tolfenamic acid for 48 hr were incubated with VEGF32P oligonucleotide alone (FIG. 3A) or in combination with various Sp antibodies (FIG. 3B) and analyzed by gel mobility shift.

FIGS. 4A-4D show non-steroidal anti-inflammatory drugs modulate VEGF expression in Panc-1 cells. FIG. 4A shows VEGF protein expression. Panc-1 cells were treated with DMSO, 50 μM ampiroxicam or tolfenamic acid for 48 hr and VEGF protein levels were determined by Western blot analysis. FIG. 4B shows VEGF mRNA levels. Panc-1 cells were treated with DMSO, 50 μM ampiroxicam and tolfenamic acid for 12 hr, and mRNA levels relative to glyceraldehyde 3-phosphate dehydrogenase (GAPDH), were determined by semi-quantitative RT-PCR. Results in (FIG. 4A) and (FIG. 4B) are presented as means ±SE for three separate determinations for each treatment group and significantly (p<0.05) decreased VEGF expression is indicated by an asterisk. FIG. 4C shows VEGF mRNA stability. Panc-1 cells were treated with actinomycin D (5 mg/ml) alone or in combination with 50 μM tolfenamic acid for up to 4.5 hr, and VEGF mRNA levels were determined at time 0 and 1.5, 2.5 and 4.5 hr after treatment by semi-quantitative RT-PCR. Results are the average of duplicate experiments, and VEGF mRNA levels are normalized to GADPH mRNA. FIG. 4D shows immunostaining of VEGF. Panc-1 cells were treated with DMSO, 50 μM ampiroxicam and tolfenamic acid for 48 hr and immunostaining for VEGF expression was determined.

FIGS. 5A-5B show effects of the proteasome inhibitor lactacystin on tolfenamic acid-induced degradation of Sp proteins and activation of pVEGF2. FIG. 5A shows that lactacystin inhibits degradation of Sp proteins. Panc-1 cells were treated with DMSO, 50 μM ampiroxicam or tolfenamic acid alone or in combination with 2 μM lactacystin for 48 hr, and whole cell lysates were analyzed for Sp proteins by Western blot analysis. FIG. 5B shows that lactacystin inhibits decreased transactivation in cells transfected with pVEGF2 construct. Panc-1 cells were transfected with pVEGF2 construct, treated with DMSO, 50 μM ampiroxicam and tolfenamic acid alone or in combination with 2 μM lactacystin, and luciferase activity determined. Results are expressed as means ±SE for three separate determinations for each treatment group and significantly (p<0.05) decreased activity by tolfenamic acid (*) or inhibition of this response by lactacystin (**) are indicated.

FIGS. 6A-6E show comparative effects of non-steroidal anti-inflammatory drugs on Panc-1 cell proliferation and Sp protein expression. Effects of tolfenamic acid (FIG. 6A), ampiroxicam (FIG. 6B), naproxen (FIG. 6C), and diclofenac sodium (FIG. 6D) on Panc-1 cell proliferation. Panc-1 cells were treated with different concentrations of non-steroidal anti-inflammatory drugs for 6 days, and the number of cells were determined on days 2, 4, and 6 by cell counting techniques. Results are expressed as means of at least three replicates for each determinations, and the SE values were <15% for all time points. Similar results were obtained using the WST assay for ampiroxicam and tolfenamic acid (data not shown). FIG. 6E shows NSAID-induced apoptosis and degradation of Sp proteins. Panc-1 cells were treated with DMSO, 150 μM ampiroxicam, 150 μM naproxen, and 50 μM tolfenamic acid for 48 hr, and levels of Sp proteins and the cleaved poly (ADP-ribose) polymerase (PARP) protein were determined in whole cell lysates by Western blot analysis. The concentrations of non-steroidal anti-inflammatory drugs used in the experiment corresponded to doses that exhibited comparable growth inhibitory activities.

FIGS. 7A-7D compares effects of tolfenamic acid and ampiroxicam on pancreatic cancer cell proliferation. L3.6pl (FIGS. 7A, 7B), and Panc-28 (FIGS. 7C, 7D) cells were treated with DMSO, 25, 50 or 100 μM ampiroxicam or tolfenamic acid for 6 days, and every 2 days, cells were counted. Results are expressed as means ±SE for three separate determinations for each treatment group and tolfenamic acid significantly (p<0.05) inhibited cell growth at concentrations of 25-100 μM.

FIGS. 8A-8E show inhibition of pancreatic tumor growth and angiogenesis by tolfenamic acid and gemcitabine. Decreased tumor volume (FIG. 8A) and weight (FIG. 8B). Median tumor volumes (FIG. 8A) and weights (FIG. 8B) in athymic nude mice treated orthotopically with L3.6pl cells, and corn oil (control), tolfenamic acid (25 and 50 mg/kg), and gemcitabine (50 mg/kg) were determined. Results are expressed as means ±SD and significantly (p<0.05) decreased tumor volumes and weights compared to the corn oil control are indicated by an asterisk. FIG. 8C shows Sp and VEGF protein expression. Tumors from the various treatment groups were analyzed for Sp1, Sp3, Sp4, and VEGF protein expression by Western blot analysis. Results are expressed as means ±SE for at least three separate determinations for each treatment group and expressed relative to the solvent (corn oil) control and normalized to β-tubulin within each group. Immunostaining is shown for VEGF (FIG. 8D), and CD31 (FIG. 8E). Pancreatic tumor sections from animals treated with solvent (control), gemcitabine (50 mg/kg) and tolfenamic acid (25 and 50 mg/kg) were immunostained with VEGF and CD31 antibodies.

FIGS. 9A-9B show Sp protein domains. FIG. 9A shows structural features of Sp protein domains that are used to prepare Gal4/Spx-y constructs. FIG. 9B shows lysine residue distribution in Sp protein. Numbers represent amino acid sites according to REFSEQ accession number for Sp proteins.

FIG. 10 shows Gal4/Spx-y constructs.

FIG. 11A-11C shows VEGF (FIG. 11A), VEGFR1 (FIG. 11B) and VEGFR2 (FIG. 11C) constructs.

FIG. 12 shows GC-rich p27 promoter construct.

FIGS. 13A-13D show VEGFR1 expression in pancreatic cancer cells is Sp protein-dependent. Transfection with pVEGFR1-A (FIG. 13A), pVEGFR1-B (FIG. 13B), and pVEGFR1-C (FIG. 13C), and small inhibitory RNAs for Sp1 (iSp1), Sp3 (iSp3), or Sp4 (iSp4) (FIG. 13D). Panc-1 cells were transfected with the pVEGFR1 constructs and iSp1, iSp3 or iSp4, and luciferase activity was determined. As a control for the transfection experiment, cells were transfected with a non-specific small inhibitory RNA (iNS) and luciferase activity for transfection with iNS was set to 100%. All experiments were replicated at least three times and results are expressed as means ±SD. Significantly (p<0.05) decreased luciferase activity is indicated (*). The effect of iSp1, iSp3, and iSp4, on Sp and VEGFR1 proteins is shown (FIG. 13D). Panc-1 cells were transfected with iNS, iSp1, iSp3, or iSp4, and whole cell lysates were analyzed by Western blot analysis. The experiment was replicated (3 times) and relative expression of individual Sp proteins and VEGFR1 were compared to levels in cells treated with iNS (set at 100%). Significantly (p<0.05) decreased protein expression is indicated by an asterisk.

FIGS. 14A-14D show tolfenamic acid decreases expression of Sp and VEGFR1 proteins in pancreatic cancer cells. Tolfenamic acid effects on Panc-1 cells (FIG. 14A and FIG. 14B), and L3.6pl cells (FIG. 14C and FIG. 14D). Cells were treated with DMSO, 50 μM tolfenamic acid, or 50 μM ampiroxicam for 48 h, and whole cell lysates were analyzed by Western blot analysis. The experiment was replicated (3 times), and the Sp and VEGFR1 protein levels were set at 100% and significantly (p<0.05) decreased expression of Sp1, Sp3, Sp4, and VEGFR2, is indicated by an asterisk.

FIG. 15 shows immunohistochemical analysis of VEGFR1 in pancreatic cancer cells treated with tolfenamic acid. Panc-1 and L3.6pl cells were treated with DMSO, 50 μM ampiroxicam, or 50 μM tolfenamic acid for 48 h, and cells were immunostained with VEGFR1 antibodies.

FIGS. 16A-16D show the interaction of Sp proteins with VEGFR1 promoter sequences. Panc-1 cells were treated with DMSO, 50 μM ampiroxicam, or 50 μM tolfenamic acid for 48 h, and nuclear extracts were incubated with VEGFR1³²P (FIG. 16A) or GC/SP³²P (FIG. 16B) oligonucleotides in the presence or absence of Sp1, Sp3, or Sp4 antibodies and analyzed in a gel mobility shift assay. The specific Sp protein bands and antibody supershifted complexes are indicated with arrows. Effects of tolfenamic acid on VEGFR1 and Egr-1 protein expression in Panc-1 cells (FIG. 16C), and L3.6pl cells (FIG. 16D). Cells were treated with DMSO, 50 μM ampiroxicam, or 50 μM tolfenamic acid for 48 h, and whole cell lysates were examined by Western blot analysis.

FIGS. 17A-17D show effects of tolfenamic acid on the VEGFR1 promoter and mRNA expression. Transfection with pVEGFR1-A (FIG. 17A), pVEGFR1-B (FIG. 17B), and pVEGFR1-C (FIG. 17C). Panc-1 cells were transfected with pVEGFR1 constructs, treated with DMSO, 50 μM ampiroxicam, or 50 μM tolfenamic acid, and luciferase activity was determined as described in Materials and Methods. Results are expressed as means ±SD for three replicate experiments for each treatment group and significantly (p<0.05) decreased activity is indicated by an asterisk. FIG. 17D shows decreased VEGFR1 mRNA in Panc-1 and L3.6pl cells. Panc-1 or L3.6pl cells were treated with DMSO, 50 μM ampiroxicam, or 50 μM tolfenamic acid for 24 h, and relative mRNA expression was determined by semi-quantitative RT-PCR. Results are shown for a single experiment and similar data was obtained in replicate experiments.

FIGS. 18A-18D show tolfenamic acid inhibits activation of VEGFR1 in pancreatic cancer cells. Inhibition of Erk1/2 phosphorylation in Panc-1 (FIG. 18A), and L3.6pl (FIG. 18B) cells. Cells were pretreated with DMSO, 50 μM ampiroxicam, or 50 μM tolfenamic acid for 36 hr; VEGF-B (50 ng/ml) was added for 5 or 10 min, and whole cell lysates were obtained and analyzed by Western blot. Cell migration assay (FIG. 18C). Panc-1 cells were treated with DMSO, 50 μM ampiroxicam, or 50 μM tolfenamic acid for 24 h. VEGF-B (50 ng/ml) was added and the inhibition of cell migration (relative to DMSO-treated cells set at 100%) was determined 16 h after addition of VEGF-B. The experiments were replicated three times, and results are expressed as means ±SD and significantly (p<0.05) decreased cell migration is indicated (*). Immunostaining of pancreatic tumors (FIG. 18D). Pancreatic tumors from athymic nude mice treated with solvent (control), gemcitabine (50 mg/kg), or tolfenamic acid (25 and 50 mg/kg) (Dick, 1996) were stained with VEGFR1 antibodies as described below and in a previous report (Dick, 1996).

FIGS. 19A-19B show tolfenamic acid inhibits growth of SEG-1 (FIG. 19A) and BIC-1 (FIG. 19B) esophageal cancer cells.

FIGS. 20A-20B show tolfenamic acid (TA) decreases expression of Sp1, Sp3, and Sp4 proteins in SEG-1 (FIG. 20A) and BIC-1 (FIG. 20B) esophageal cancer cells.

FIGS. 21A-21B show tolfenamic acid (TA) decreases expression of Sp1, Sp3, and Sp4 and Sp-dependent proteins survivin and vascular endothelial growth factor (VEGF), and induces apoptosis as shown by PARP cleavage (FIG. 21A), and decreases expression of Sp1, Sp3, and Sp4 and Sp-dependent protein c-Met (FIG. 21B), in SEG-1 and BIC-1 esophageal cancer cells.

### DETAILED DESCRIPTION OF THE INVENTION

The present invention demonstrates that chemical-induced degradation of Sp proteins by the diphenyl/diphenylamine carboxylic acid subclass of non-steroidal anti-inflammatory drugs inhibited growth and angiogenesis in cancer cells. The present invention demonstrates tolfenamic acid and structurally related non-steroidal anti-inflammatory drugs specifically activate proteosome-dependent degradation of Sp family transcription factors (Sp1, Sp3 and Sp4) in cancer cells. Further the present invention demonstrates inhibition of cell proliferation through induction of p27, and decreased Sp-dependent expression of the angiogenic factor, vascular endothelial growth factor. These are the only compounds that induced degradation of Sp1, Sp3, and Sp4. In vitro and in vivo experiments demonstrate the compounds of the present invention inhibit pancreatic cell and tumor growth, pancreatic tumor angiogenesis, and liver metastasis, and esophageal cancer cell growth and induction of apoptosis.

Vascular endothelial growth factor and related angiogenic growth factors and their receptors play a critical role in tumorigenesis and contribute significantly to cancer cell progression and metastasis. Not surprisingly, vascular endothelial growth factor/vascular endothelial growth factor receptor (VEGFR) signaling pathways have been extensively targeted for cancer chemotherapy. Anti-angiogenic compounds initially discovered have multiple mechanisms of action; however, several alternative approaches have also been reported and these include antibodies that block vascular endothelial growth factor and/or vascular endothelial growth factor receptor, and tyrosine kinase inhibitors that block vascular endothelial growth factor receptor kinase signaling. Other approaches include development of arginine-rich peptides that block vascular endothelial growth factor action or by blocking downstream factors such as Src family kinases that mediate some of the VEGFR1-dependent responses in colon cancer cells. A construct expressing domains of both VEGFR1 and VEGFR2 that tightly binds vascular endothelial growth factor through the extra-cellular domain of VEGFR-1 (VEGF-Trap) has also been used to inhibit tumor growth and metastasis in animal models.

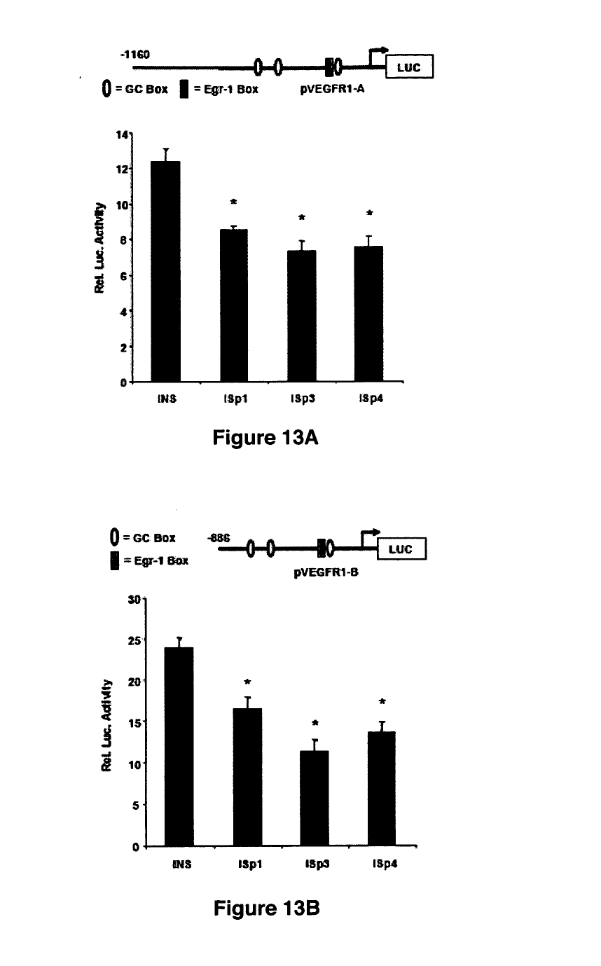

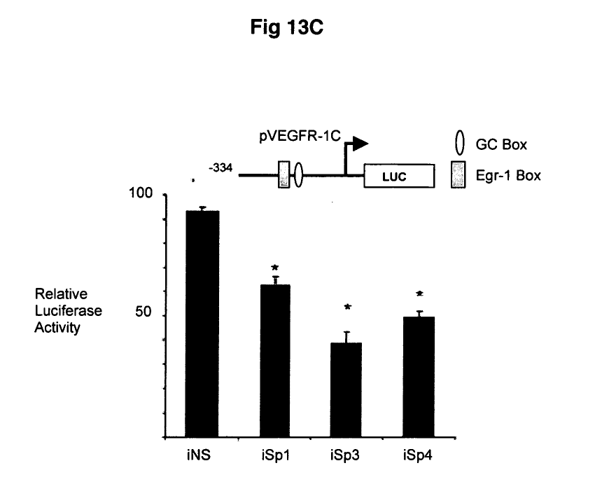

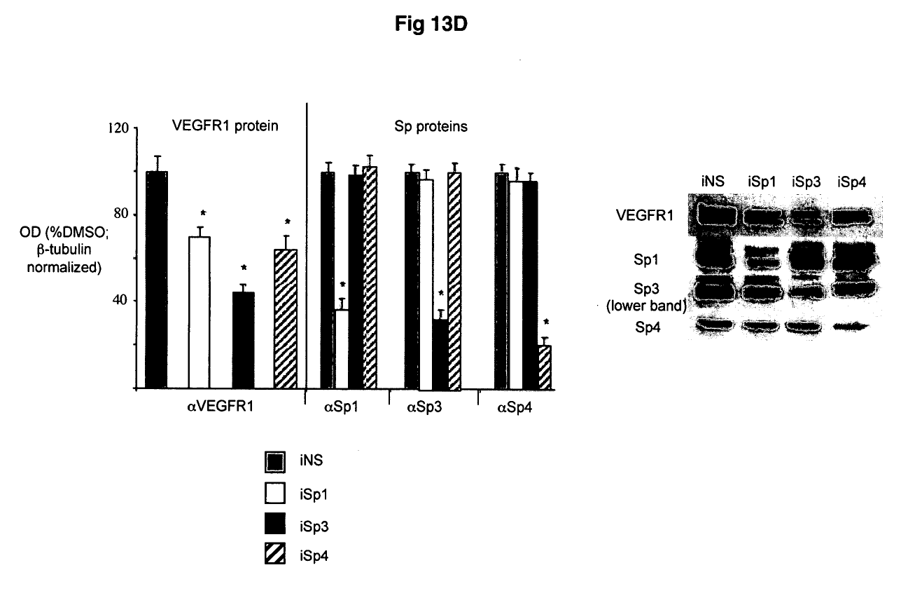

Previous studies have shown that expression of both vascular endothelial growth factor and VEGFR2 in pancreatic cancer cells, and other cancer cell lines was regulated by Sp1, Sp3, and Sp4; and RNA interference with small interfering RNAs targeting these proteins decreased vascular endothelial growth factor and VEGFR2 expression. The instant invention has demonstrated in pancreatic cancer cells, ad esophageal cancer cells, and tumors of these cancers, that tolfenamic acid induced proteasome-dependent degradation of Sp1, Sp3, and Sp4; and not surprisingly, tolfenamic acid inhibited angiogenesis and decreased liver metastasis in an orthotopic model of pancreatic cancer using the highly metastatic L3.6pl cell. Since VEGFR1 also plays a pivotal role in pancreatic tumor migration and invasion, the present invention investigated the molecular mechanism of VEGFR1 regulation in pancreatic cancer cells. Takeda and coworkers identified VEGFR1 as a gene regulated by the basic helix-loop-helix endothelial PAS domain protein 1 (EPAS1), which forms a heterodimer with hypoxia-inducible factor 1B to activate VEGFR1 and other pro-angiogenic genes. However, the VEGFR1 gene has three consensus GC-rich motifs that bind Sp proteins, and results from RNA interference studies show that knockdown of Sp1, Sp3, or Sp4 also decreased VEGFR1 protein expression in pancreatic cancer cells (FIG. 13). Moreover, similar results were observed using a series of deletion constructs containing VEGFR1 promoter inserts (FIG. 13).

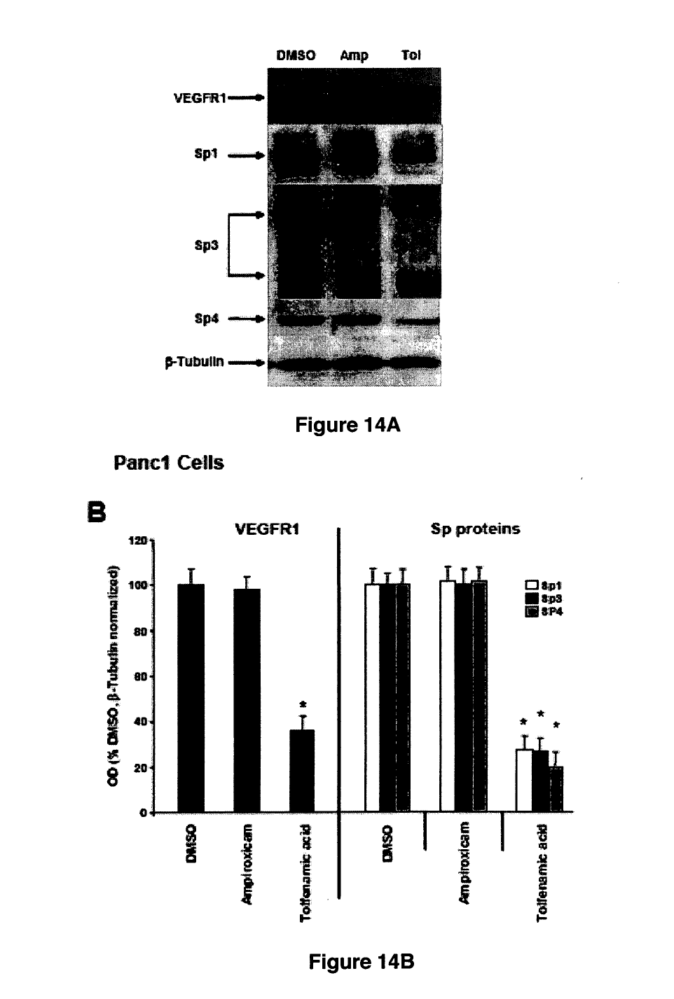

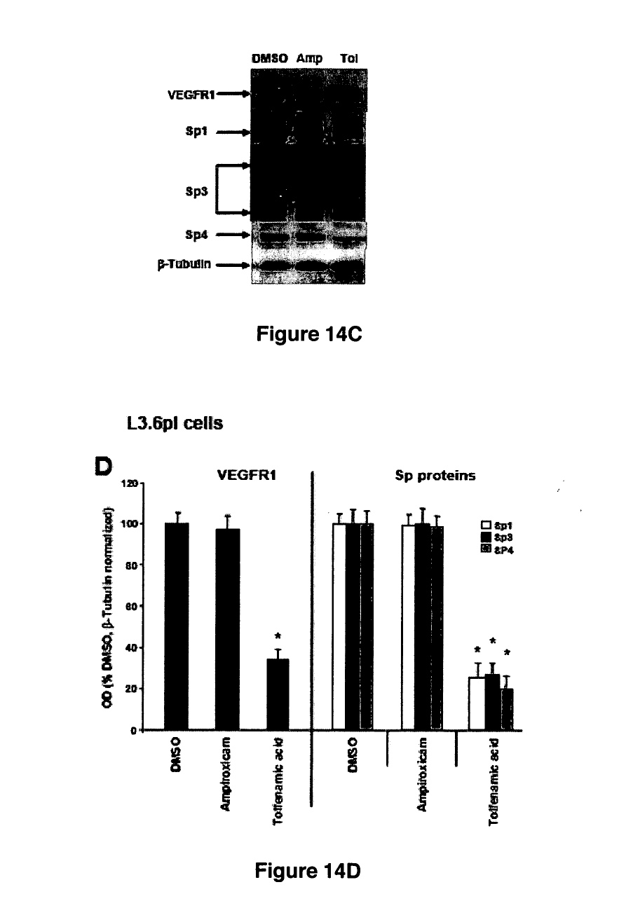

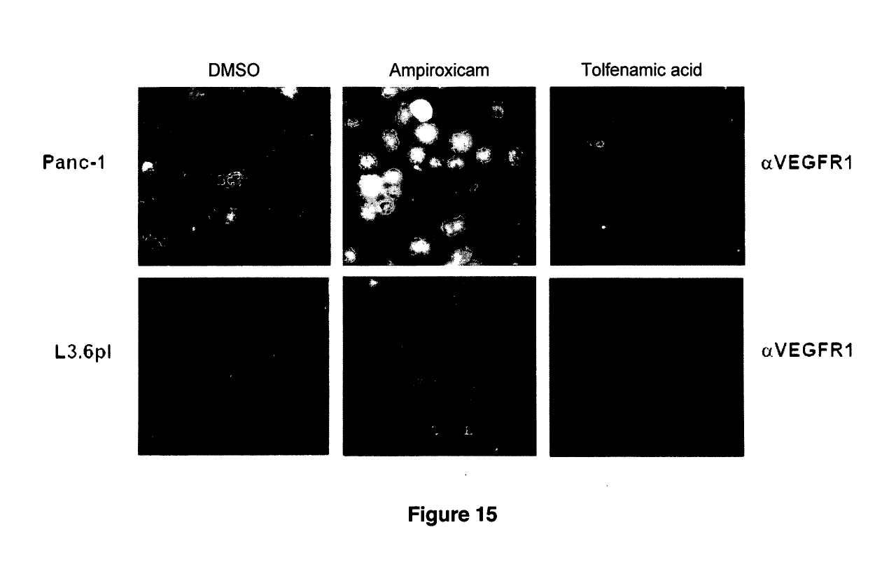

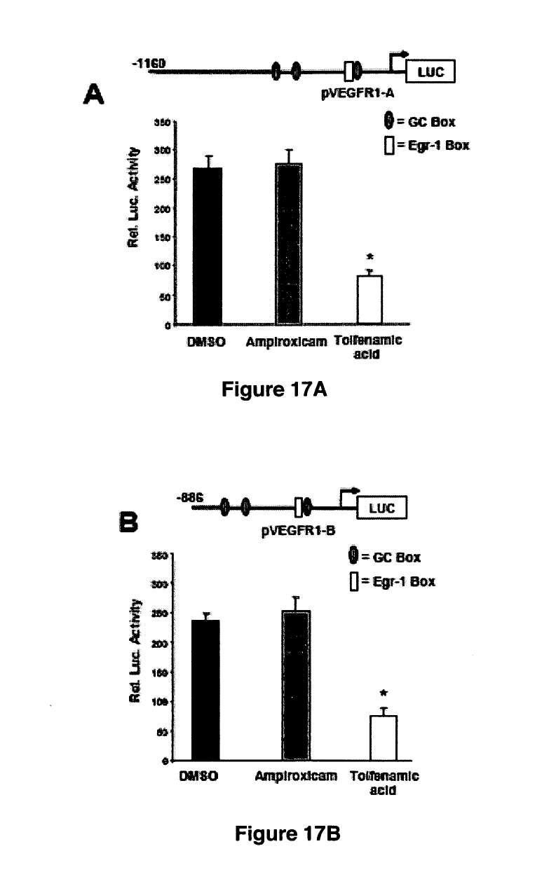

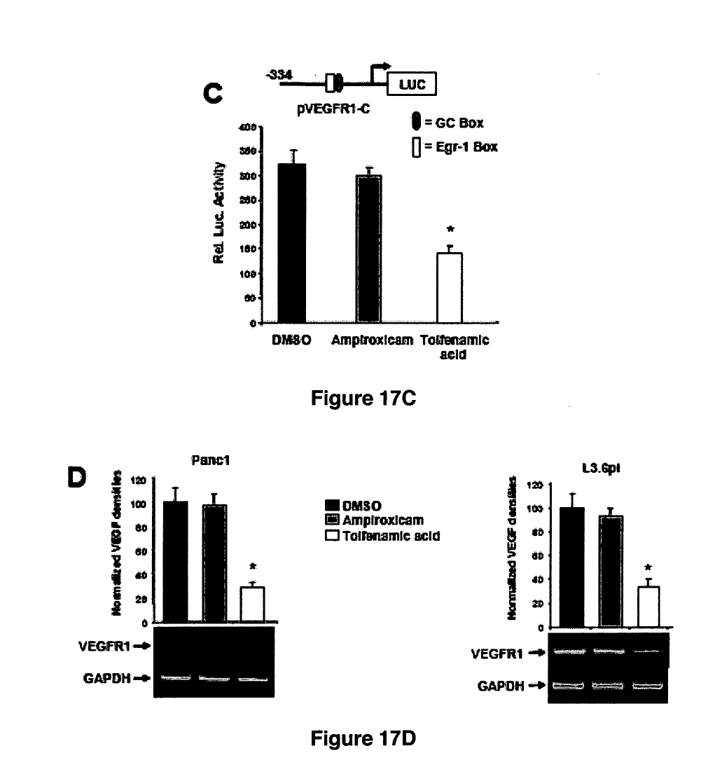

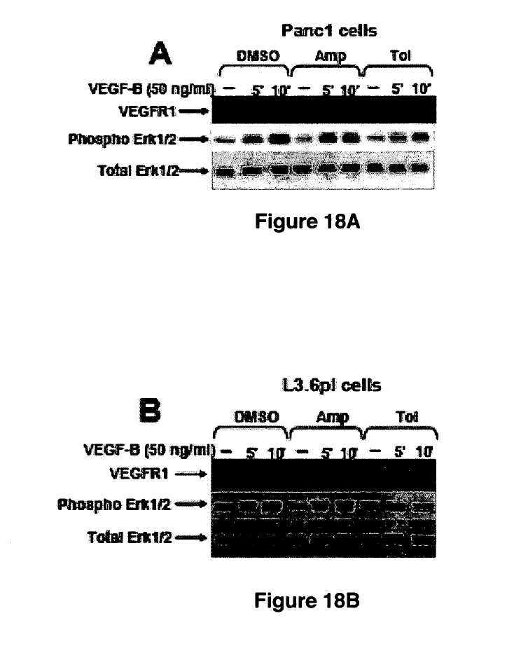

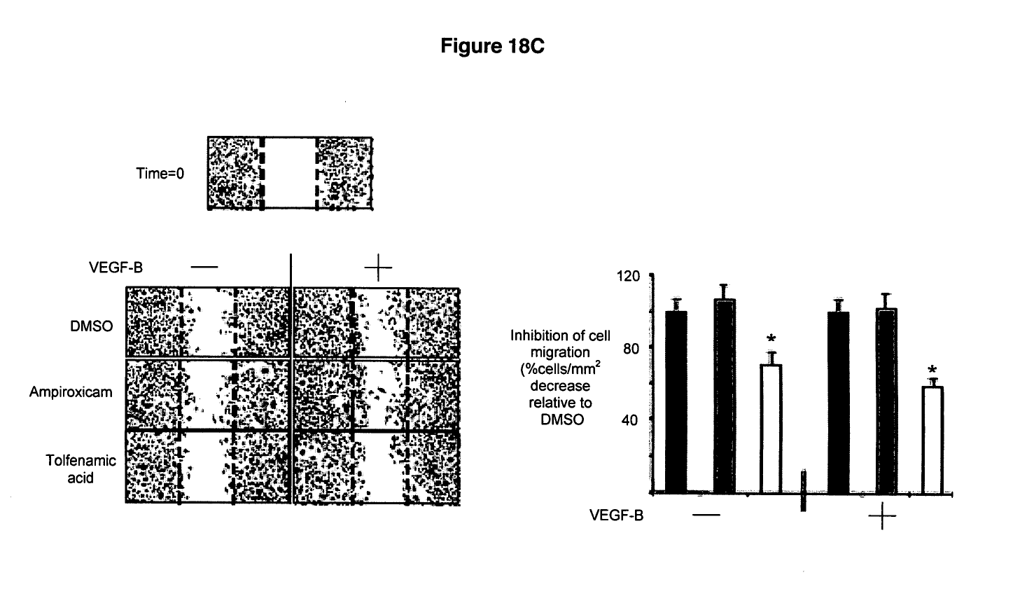

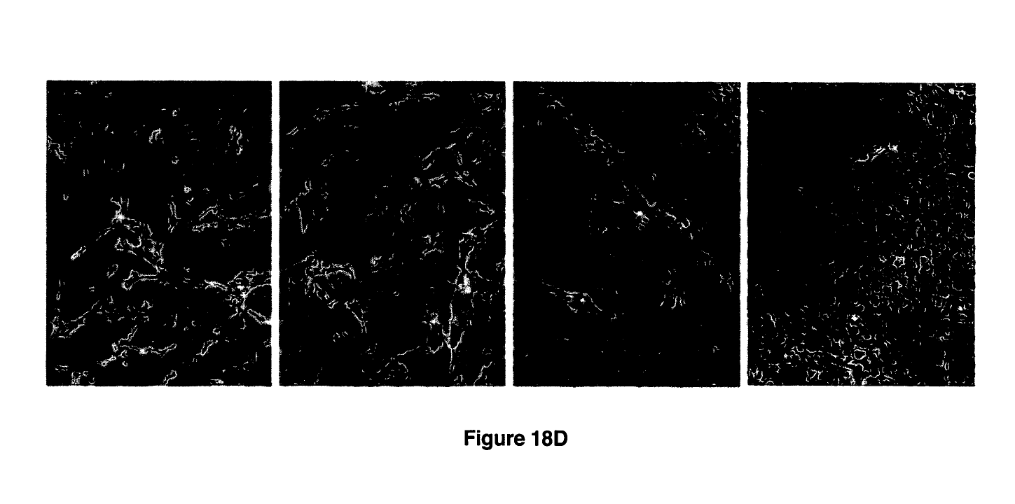

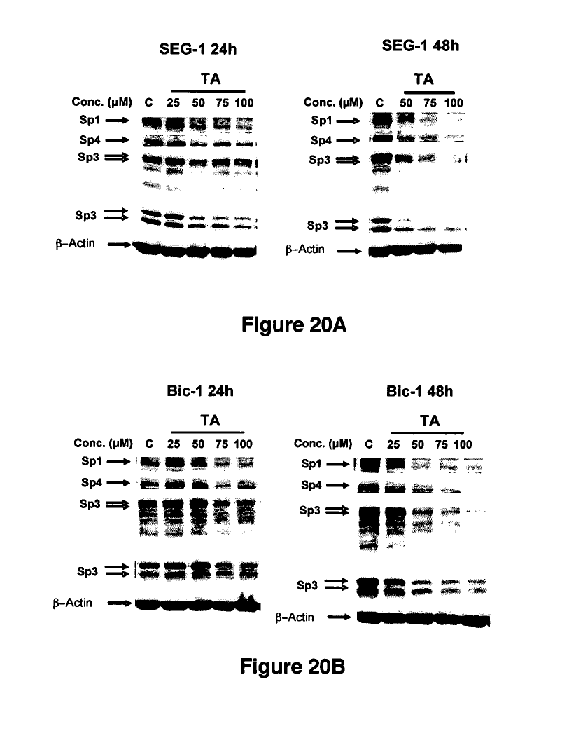

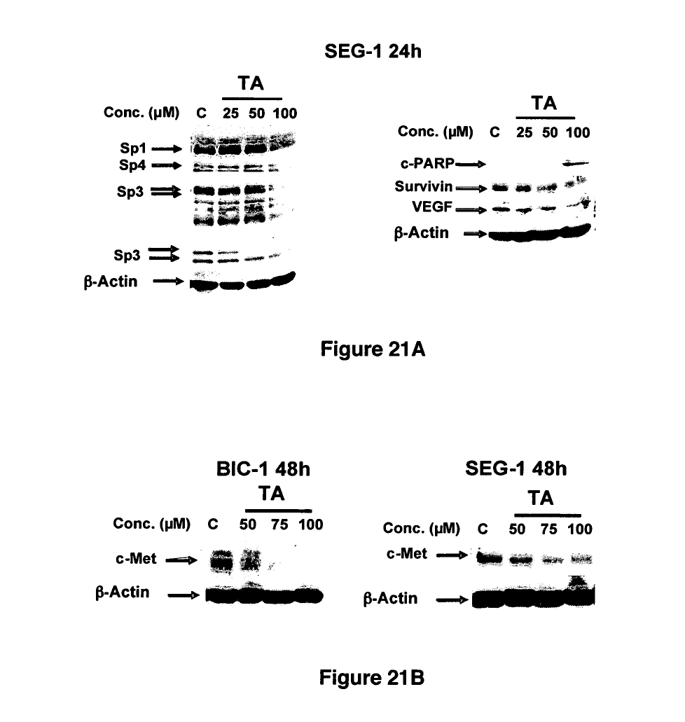

These results suggest that like vascular endothelial growth factor and vascular endothelial growth factor receptor, VEGFR1 expression in pancreatic cancer cells is Sp-dependent, and therefore compounds such as tolfenamic acid, which decrease Sp1, Sp3, and Sp4 (FIG. 15, FIGS. 20A-20B, and FIGS. 21A-21B) should also decrease VEGFR1. This was confirmed in a series of experiments showing that tolfenamic acid but not the non-steroidal anti-inflammatory drugs ampiroxicam, a negative control, decreases VEGFR1 protein (FIGS. 14A, 14C, 15, and 16), and mRNA (FIG. 17D), as well as luciferase activity in cells transfected with VEGFR1 constructs (FIGS. 17A-17B). Moreover, decreased VEGFR1 expression (FIG. 18D) was observed in pancreatic tumors from mice treated with tolfenamic acid, and this was accompanied by decreased levels of vascular endothelial growth factor and Sp protein in these tumors. Since VEGFR1 mediates vascular endothelial growth factor B (VEGFB)-induced migration and invasion of pancreatic and colon cancer cells, we also investigated the role of Sp proteins in mediating Panc-1 cell migration in a “scratch” test in monolayer cultures grown on collagen IV coated plates (FIG. 18C). Previous reports show that VEGF-A and VEGF-B induce migration of colon and pancreatic cancer cells using a Boyden chamber assay, and results in FIG. 18C confirm that VEGF-B also induced migration of Panc1 cells. Inhibition of VEGF-B-induced migration of colon and pancreatic cancer cells was observed in cells treated with the VEGFR1 antibody (18F1), and similar inhibitory effects were observed in this study in Panc1 cells co-treated with VEGF-B plus tolfenamic acid (FIG. 18C). In contrast, tolfenamic acid also inhibited Panc1 cell migration in solvent-treated (DMSO) cells, whereas the VEGFR1 antibody did not affect migration of untreated/solvent control cells. The differences between tolfenamic acid and VEGFR1 antibodies must be due to the overall decrease in VEGFR1 levels (FIG. 14) in cells treated with tolfenamic acid, whereas the antibodies do not affect VEGFR1 expression. VEGF-B-induced phosphorylation of MAPK was also examined in Panc1 and L3.6pl cells and, like the VEGFR1 antibody, tolfenamic acid decreased MAPK phosphorylation in Panc1 and L3.6pl cells (FIGS. 18A and 18B). This was consistent with the parallel down regulation of VEGFR1 protein in these cells. Thus, the inhibition of VEGF-B-induced migration of pancreatic cancer cells by tolfenamic acid is also accompanied by inhibition of VEGFR1-dependent downstream signaling. Ampiroxicam, a non-steroidal anti-inflammatory drug that does not induce Sp protein degradation in Panc1 cells, was also used as a control in the cell migration and MAPK phosphorylation studies, and this compound was inactive in all assays.

The results discussed herein show that cancer cell growth and angiogenesis can be targeted via the Sp proteins. Furthermore, although the inhibitory effects of these compounds were demonstrated in pancreatic cancer cells and pancreatic tumors, and esophageal cancer cells, similar effects were also observed in breast, prostate, colon, bladder, and ovarian cancer cells. Overall, tolfenamic acid and structurally-related non-steroidal anti-inflammatory drugs containing diphenyl and diphenylamine carboxylic acid structures alone or in combination with other cancer chemotherapeutic drugs provides the advantage of using relatively non-toxic well-tested drugs for inhibiting tumor growth and angiogenesis by degrading all three Sp proteins (Sp1, Sp3, and Sp4) that are critical for tumor/cancer cell growth and metastasis.

In one embodiment of the present invention, there is provided a method of inducing degradation of one or more Sp transcription factors, comprising: contacting a cancer cell with a non-steroidal anti-inflammatory drug thereby inducing degradation of one or more Sp transcription factors. The degradation of Sp transcription factors may induce the expression of p27 such that proliferation of the cancer cell is inhibited. The degradation may also decrease the expression of vascular endothelial growth factor such that angiogenesis, tumor metastasis, or a combination thereof, in a tumor comprising the cancer cell is inhibited. Preferably, the non-steroidal anti-inflammatory drug is a diphenyl/diphenylamine carboxylic acid. Representative examples of such diphenyl/diphenylamine carboxylic acid includes but is not limited to tolfenamic acid, diclofenac sodium, and diflunisal. Moreover, the Sp transcription factor is a Sp1 protein, Sp3 protein, or Sp4 protein, or a combination thereof. Additionally, examples of the cancer cell include but are not limited to a pancreatic cancer cell, an esophageal cancer cell, a breast cancer cell, a prostate cancer cell, a colon cancer cell, a bladder cancer cell, or an ovarian cancer cell.

In another embodiment of the present invention, there is provided a method of treating cancer in an individual, comprising: administering a pharmacologically effective amount of diphenyl/diphenylamine carboxylic acid to the individual, thereby treating the cancer in the individual. This method may further comprise administering another chemotherapeutic drug. Such a chemotherapeutic drug may be administered concurrently or sequentially with the diphenyl/diphenylamine carboxylic acid. Furthermore, such a diphenyl/diphenylamine carboxylic acid may inhibit tumor growth, may inhibit angiogenesis, and inhibit metastasis in the individual. Examples of such diphenyl/diphenylamine carboxylic acid include but are not limited to tolfenamic acid, diclofenac sodium, and diflunisal. Additionally, an individual benefiting from such a method includes but is not limited to one who is diagnosed with pancreatic cancer, esophageal cancer, breast cancer, prostate cancer, colon cancer, bladder cancer, or ovarian cancer.

In yet another embodiment of the present invention, there is provided a method of treating pancreatic cancer in an individual, comprising: administering a pharmacologically effective amount of tolfenamic acid to the individual, where the tolfenamic acid inhibits proliferation, angiogenesis and metastasis of the pancreatic cancer, thereby treating the pancreatic cancer in the individual. This method may further comprise administering another chemotherapeutic drug. Such a chemotherapeutic drug may be administered concurrently or sequentially with the tolfenamic acid.

In yet another embodiment of the present invention, there is provided a method of treating esophageal cancer in an individual, comprising: administering a pharmacologically effective amount of tolfenamic acid to the individual, where the tolfenamic acid inhibits proliferation, angiogenesis and metastasis of the esophageal cancer, thereby treating the esophageal cancer in the individual. This method may further comprise administering another chemotherapeutic drug. Such a chemotherapeutic drug may be administered concurrently or sequentially with the tolfenamic acid.

In another embodiment of the present invention, there is provided a method of reducing toxicity of a cancer therapy in an individual in need thereof, comprising: administering to the individual a diphenyl/diphenylamine carboxylic acid and another chemotherapeutic drug, where the dosage of the chemotherapeutic drug administered is lower than the dosage required when the chemotherapeutic drug is administered singly, thereby reducing the toxicity of the cancer therapy in the individual. The chemotherapeutic drug may be administered concurrently or sequentially with the diphenyl/diphenylamine carboxylic acid. Examples of such chemotherapeutic drug and diphenyl/diphenylamine carboxylic acid include and are not limited to tolfenamic acid, diclofenac sodium, and diflunisal. Furthermore, examples of the cancer include and are not limited to pancreatic cancer, esophageal cancer, breast cancer, prostate cancer, colon cancer, bladder cancer, or ovarian cancer.

In yet another embodiment of the present invention there is a method of treating cancer in an individual, consisting of administering a pharmacologically effective amount of diphenyl/diphenylamine carboxylic acid; and a small interfering RNA specific for one or more Sp transcription factors, to the individual, thereby treating the cancer in the individual. In general, the treatment inhibits tumor growth, angiogenesis, and/or metastasis in the individual. Specifically, the diphenyl/diphenylamine carboxylic acid is tolfenamic acid, diclofenac sodium, or diflunisal. The individual is diagnosed with pancreatic cancer, esophageal cancer, breast cancer, prostate cancer, colon cancer, bladder cancer, or ovarian cancer. Specifically, the Sp transcription factor is a Sp1 protein, a Sp3 protein, a Sp4 protein, or a combination thereof.

In still yet another embodiment of the present invention there is a method of inhibiting the angiogenic response of a tumor in an individual consisting of administering a pharmacologically effective amount of diphenyl/diphenylamine carboxylic acid; and a small interfering RNA specific for one or more Sp transcription factors, to the individual, thereby inhibiting the angiogenic response of the tumor in said individual. In general, this method results in decrease in expression of VEGFR1, such that the angiogenic response of the tumor is inhibited. Moreover, this method inhibits tumor growth and metastasis in the individual. Specifically, the diphenyl/diphenylamine carboxylic acid is tolfenamic acid, diclofenac sodium, or diflunisal. In general, the individual has a pancreatic cancer, esophageal cancer, breast cancer, prostate cancer, colon cancer, bladder cancer, or ovarian cancer tumor.

In still yet another embodiment of the present invention there is a method of inhibiting tumor metastasis in an individual consisting of administering a pharmacologically effective amount of diphenyl/diphenylamine carboxylic acid; and a small interfering RNA specific for one or more Sp transcription factors, to the individual, thereby inhibiting tumor metastasis in said individual. In general, the method decreases the expression of VEGFR1, such that tumor metastasis is inhibited. Specifically, the diphenyl/diphenylamine carboxylic acid is tolfenamic acid, diclofenac sodium, or diflunisal. In general, the individual has a pancreatic cancer, esophageal cancer, breast cancer, prostate cancer, colon cancer, bladder cancer, or ovarian cancer tumor.

As used herein, the term, “a” or “an” may mean one or more. As used herein in the claim(s), when used in conjunction with the word “comprising”, the words “a” or “an” may mean one or more than one. As used herein “another” or “other” may mean at least a second or more of the same or different claim element or components thereof.

As used herein, the term “contacting” refers to any suitable method of bringing a non-steroidal anti-inflammatory drug into contact with a cancer cell. In vitro or ex vivo, this is achieved by exposing the cancer cell to the non-steroidal anti-inflammatory drug in a suitable medium. For in vivo application, any known method of administration is suitable as described herein.

As used herein, the term “treating” or the phrase “treating a cancer” includes, but is not limited to, halting the growth of the cancer, killing the cancer, or reducing the size of the cancer. Halting the growth refers to halting any increase in the size, or the number of, or size of the cancer cells, or to halting the division of the neoplasm, or the cancer cells. Reducing the size refers to reducing the size of the cancer, or the number of, or size of the cancer cells.

Another chemotherapeutic drug may be administered concurrently or sequentially with the non-steroidal anti-inflammatory drug used herein. The effect of co-administration with the non-steroidal anti-inflammatory drug is to lower the dosage of the chemotherapeutic drug normally required that is known to have at least a minimal pharmacological or therapeutic effect against a cancer or cancer cell, for example, the dosage required to eliminate a cancer cell. Concomitantly, toxicity of the chemotherapeutic drug to normal cells, tissues, and organs is reduced without reducing, worsening, eliminating, or otherwise interfering with any cytotoxic, cytostatic, apoptotic or other killing or inhibitory therapeutic effect of the drug on the cancer cells.

Non-steroidal anti-inflammatory drugs used herein and other chemotherapeutic drugs can be administered independently, either systemically or locally, by any method standard in the art, for example, subcutaneously, intravenously, parenterally, intraperitoneally, intradermally, intramuscularly, topically, enterally, rectally, nasally, buccally, vaginally, or by inhalation spray, by drug pump, or contained within a transdermal patch, or an implant. Dosage formulations of the non-steroidal anti-inflammatory drugs may comprise conventional non-toxic, physiologically or pharmaceutically acceptable carriers, or vehicles suitable for the method of administration.

The non-steroidal anti-inflammatory drugs and chemotherapeutic drugs or pharmaceutical compositions thereof may be administered independently one or more times to achieve, maintain or improve upon a therapeutic effect. It is well within the skill of an artisan to determine dosage or whether a suitable dosage of either or both of the non-steroidal anti-inflammatory drugs and chemotherapeutic drug comprises a single administered dose or multiple administered doses. An appropriate dosage depends on the health of the individual, the progression, or remission of the cancer, the route of administration, and the formulation used.

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### EXAMPLE 1

### Cell Lines, Chemicals, Biochemical, Constructs and Oligonucleotides

Panc-1 cells were obtained from the American Type Culture Collection (ATCC®, Manassas, Va.). L3.6pl cell line was developed at M. D. Anderson Cancer Center (Houston, Tex.) and provided by Dr. I. Fidler. Esophageal cancer cells SEG-1 and Bic-1 were obtained and maintained. VEGFR1 promoter luciferase constructs were provided by Dr. Koji Maemura (University of Tokyo, Japan). DME/F12 with and without phenol red, 100× antibiotic/antimycotic solution and lactacystin were purchased from Sigma Chemical Co. (St. Louis, Mo.). Collagen IV-coated plates were purchased from Becton Dickinson Labware (Bedford, Mass.). DIFF QUIK staining kit was obtained from Dade Behring (Newark, Del.). Fetal bovine serum was purchased from Intergen (Purchase, N.Y.). [γ-³²P]ATP (300 Ci/mmol) was obtained from Perkin Elmer Life Sciences. Poly (dI-dC) and T4 polynucleotide kinase were purchased from Roche Molecular Biochemicals (Indianapolis, Ind.). Antibodies for Sp1 (PEP2), Sp3 (D-20), Sp4 (V-20), HDAC, VEGF (147), survivin (FL-142), c-Met, b-tubulin, and VEGFR1 (C-17) proteins were obtained from Santa Cruz Biotechnology (Santa Cruz, Calif.). Antibodies for ERK1/2 and pERK12/were obtained from Zymed Laboratories Inc. (San Francisco, Calif.). Cleaved PARP (ASP 214) antibody was purchased from Cell Signaling Technology (Danvers, Mass.). Monoclonal b-actin antibody was purchased from Sigma-Aldrich. Lysis buffer and luciferase reagent were obtained from Promega Corp. (Madison, Wis.). Horseradish peroxidase substrate for Western blot analysis was obtained from NEN Life Science products (Boston, Mass.). LIPOFECTAMINE™ and LIPOFECTAMINE™ 2000 were purchased from Invitrogen (Carlsbad, Calif.). b-Galactosidase reagent was obtained from Tropix (Bedford, Mass.).

### EXAMPLE 2

### Transfection of Pancreatic Cancer Cells and Preparation of Nuclear Extracts

Cells are cultured in 6-well plates in 2 ml of DME/F12 medium supplemented with 5% fetal bovine serum. After 16-20 hr when cells are 50-60% confluent, reporter gene constructs are transfected using LIPOFECTAMINE™ reagent (Invitrogen, Carlsbad, Calif.). The effects of the selective non-steroidal anti-inflammatory drugs on transactivation are investigated in Panc1 and L3.6pl cells co-transfected with different VEGF constructs (500 ng). Cells are treated with DMSO (control) or with the indicated concentration of non-steroidal anti-inflammatory drugs for 24 and/or 48 hr, then luciferase activity of lysates (relative to β-galactosidase activity) are determined. For proteasome inhibitor experiments, cells will be co-treated with 2 μM lactacystin, and for electrophoretic mobility shift assay (EMSA), nuclear extracts from Panc1 and L3.6pl cells are isolated as described, and aliquots will be stored at −80° C. until used (Abdelrahim et al, 2004; Abdelrahim et al, 2005).

### EXAMPLE 3

### Western Immunoblot

Cells are washed once with phosphate buffered saline PBS and collected by scraping in 200 μL of lysis buffer [50 mM HEPES, 0.5M sodium chloride, 1.5 mM magnesium chloride, 1 mM EGTA, 10% (v/v) glycerol, 1% Triton X-100, 5 μL/ml of Protease Inhibitor Cocktail (Sigma)]. The lysates from the cells are incubated on ice for 1 hr with intermittent vortexing followed by centrifugation at 40,000 g for 10 min at 4° C. Equal amounts of protein (60 μg) from each treatment group are diluted with loading buffer, boiled and loaded onto 10 and 12.5% SDS-polyacrylamide gel. For vascular endothelial growth factor immunoblots, 100 μg of protein are used. Samples are electrophoresed, transferred to membranes, and proteins detected by incubation with polyclonal primary antibodies Sp1, Sp3, Sp4, HDAC, VEGF (a-20), c-Met, surviving, VEGF, cleaved PARP, and β-tubulin (H-235) followed by blotting with appropriate horseradish peroxidase-conjugated secondary antibody as previously described (Abdelrahim et al, 2004). After autoradiography, band intensities are determined by a scanning laser densitometer (Sharp Electronics Corporation, Mahwah, N.J.) using ZERO-D SCANALYTICS software (Scanalytics Corporation, Billerica, Mass.).

### EXAMPLE 4

### Electrophoretic Mobility Shift Assay (EMSA)

Vascular endothelial growth factor and VEGFR1 oligonucleotides are synthesized and annealed, and 5 pmol aliquots are 5′-end-labeled using T4 kinase and [y-³²P]ATP. After the reaction sample was incubated 20 min on ice, and antibodies against Sp1, Sp3, and Sp4 proteins are added and incubated another 20 min on ice. Protein-DNA complexes are resolved by 5% polyacrylamide gel electrophoresis as previously described (Abdelrahim et al, 2004; Abdelrahim et al, 2005, Abdelrahim et al, submitted 2005). Specific DNA-protein and antibody-supershifted complexes are observed as retarded bands in the gel.

### EXAMPLE 5

### Cell Proliferation Assay

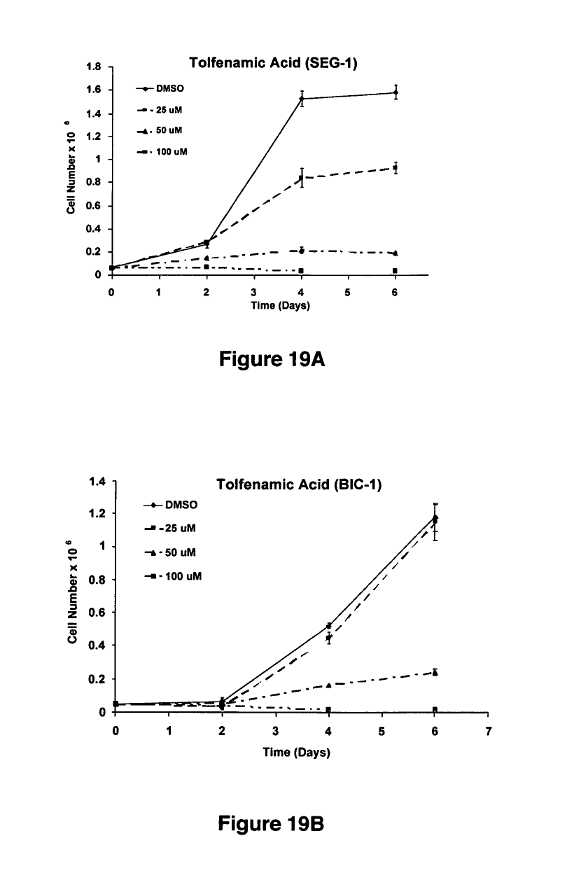

Panc1, Panc28 and L3.6pl cells are seeded in DMEM/F12 media with 5% FBS and treated the next day with either vehicle (DMSO) or with the indicated compounds' concentrations. Cells are counted at the indicated times using a Z1™ COULTER COUNTER® cell counter. Each experiment is carried out in triplicate and results expressed as means ±SD for each determination. Similar cell proliferation assays were carried out using SEG-1 and Bic-1 esophageal cancer cells and 25-100 mM tolfenamic acid concentrations with DMSO treatment as control (FIG. 19A-19B).

### EXAMPLE 6

### VEGF-B Activation of MAPK and Cell Migration

Panc-1 cells were pretreated with DMSO, 50 μM ampiroxicam, or 50 μM tolfenamic acid for 48 h, and then were treated with VEGF-B (50 ng/ml) for 5 and 10 min. MAPK phosphorylation in the various treatment groups was then determined by western immunoblot analysis as described above. For migration assays, Panc-1 cells were seeded in triplicate in six-well collagen IV coated plates and then treated with the selected non-steroidal anti-inflammatory drugs for 24 h before the scratch was made. A scratch through the central axis of the plate was gently made using a sterile pipette tip. Cells were 70% confluent when the scratch was made. Cells were washed and treated with the DMSO control, selected non-steroidal anti-inflammatory drugs alone, or non-steroidal anti-inflammatory drugs and VEGF-B. Migration of the cells into the scratch was observed at nine pre-selected points (three points per well) at 0, 8, and 16 h. Results of this study were obtained at a 16 h time point and one plate was stained using DIFF QUIK (Dade Behring, Newark, Del.).

### EXAMPLE 7

### Immunocytochemistry

Panc1 cells are seeded in LAB-TEK® chamber slides (Nalge Nunc International, Naperville, Ill.) at 100,000 cells/well in DME/F12 medium supplemented with 5% fetal bovine serum. Cells are treated with the selected non-steroidal anti-inflammatory drugs and after 48 hr, the media chamber is detached and the remaining glass slides washed in Dulbecco's phosphate buffered saline (PBS). The immunostaining for vascular endothelial growth factor (VEGF) is determined essentially as previously described (Abdelrahim et al, 2005). Briefly, the glass slides are fixed with cold (−20° C.) methanol for 10 min and washed in 0.3% PBS/Tween for 5 min (two times) before blocking with 5% goat serum in antibody dilution buffer (stock solution: 100 ml of PBS/Tween, Ig of bovine serum albumin, 45 ml of glycerol, pH 8.0) for 1 hr at 20° C. After removal of the blocking solution, VEGF rabbit polyclonal antibody (A-20) is added in antibody dilution buffer (1:200) and incubated for 12 hr at 4° C. Slides are washed for 10 min with 0.3% Tween in 0.02 M phosphate buffered saline (three times) and incubated with fluorescein isothiocyanate-conjugated (FITC) goat anti-rabbit antibodies (1:1000 dilution) for 2 hr at 20° C. Slides are then washed for 10 min in 0.3% PBS-Tween (four times). Slides are mounted in ProLonged anti-fading medium with 4′,6-diamidino-2-phenylindole (DAPI) for nuclear counterstaining (Molecular Probes, Inc., Eugene, Or) and cover slips sealed using NAILSLICKS nail polish (Noxell Corp., Hunt Valley, Md.).

### EXAMPLE 8

### Semiquantitative Reverse Transcription-PCR Analysis

Panc1 cells are treated with DMSO (control) or with the indicated concentration of non-steroidal anti-inflammatory drugs for 48 hr before total RNA collection. Total RNA is obtained with RNAZOL B (Tel-Test, Friendswood, Tex.) according to the manufacturer's protocol. RNA concentration is measured by UV 260:280 nm absorption ratio, and 200 ng/μL RNA is used in each reaction for reverse transcription (RT)-PCR. RNA is reverse transcribed at 42° C. for 25 min using oligo d(T) primer (Promega) and subsequently PCR amplified using 2 mmol/L MgCl₂, 1 μmol/L of each gene-specific primer, 1 μmol/L dNTPs, and 2.5 units AMPLITAQ® DNA polymerase (Promega). The gene products are amplified using 22 to 25 cycles (95° C., 30 s; 56° C., 30 s; 72° C., 30 s). The sequences of the oligonucleotide primers in this study are: VEFG forward: 5′-CCA TGA ACT TTC TGC TGT CTT-3′, VEGF reverse: 5′-ATC GCA TCA GGG GCA CAC AG-3′, GAPDH forward: 5′-AATCCCATCACCATCTTCCA-3′, and GAPDH reverse: 5′-GTCATCATATTTGGCAGGTT-3′, VEGFR1 forward: 5′-TGG GAC AGT AGA AAG GGC TT-3′, VEGFR1 reverse: 5′-GGT CCA CTC CTT ACA CGA CAA-3′, GAPDH forward: 5′-AAT CCC ATC ACC ATC TTC CA-3′, GAPDH reverse: 5′-GTC ATC ATA TTT GGC AGG TT-3′.

Following amplification in a PCR EXPRESS thermal cycler (Hybaid US, Franklin, Mass.), 20 μL of each sample is loaded on a 2% agarose gel containing ethidium bromide. Electrophoresis is performed at 80V in 1× Tris-acetic acid-EDTA (TAE) buffer for 1 hr and photographed by UV transillumination using POLAROID® film (Waltham, Mass.). VEGF, GAPDH, Sp1, and Sp4 band intensity values obtained by scanning the Polaroid on a SHARP JX-330 scanner (Sharp Electronics, Mahwah, N.J.); background signal is subtracted; and densitometric analysis is performed on the inverted image using ZERO-D software (Scanalytics). Results are expressed as VEGF band intensity values normalized to GAPDH values and then by averaging three separate determinations for each treatment group. A similar approach will be used for VEGFR1, VEGFR2, and other angiogenic factors.

### EXAMPLE 9

### Plasmids and Constructs

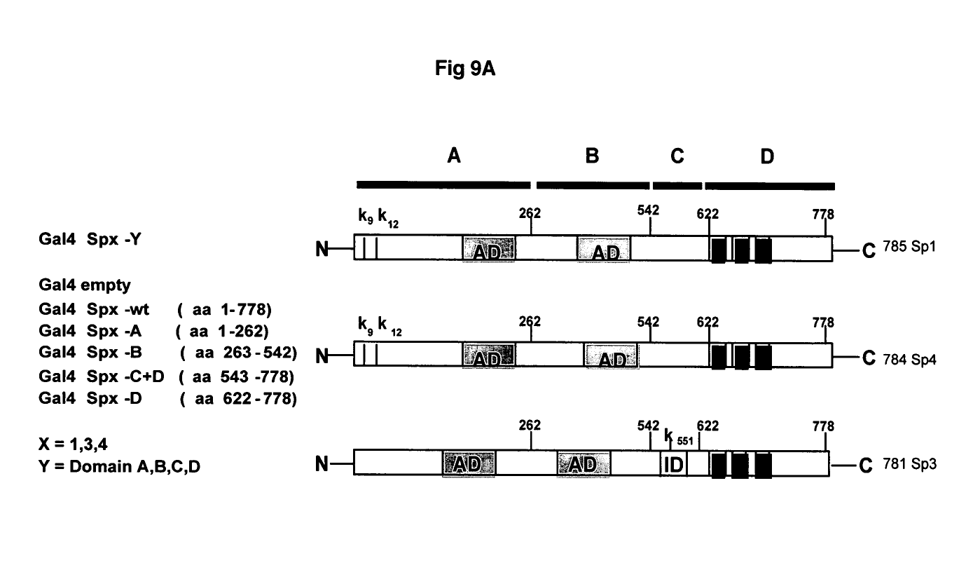

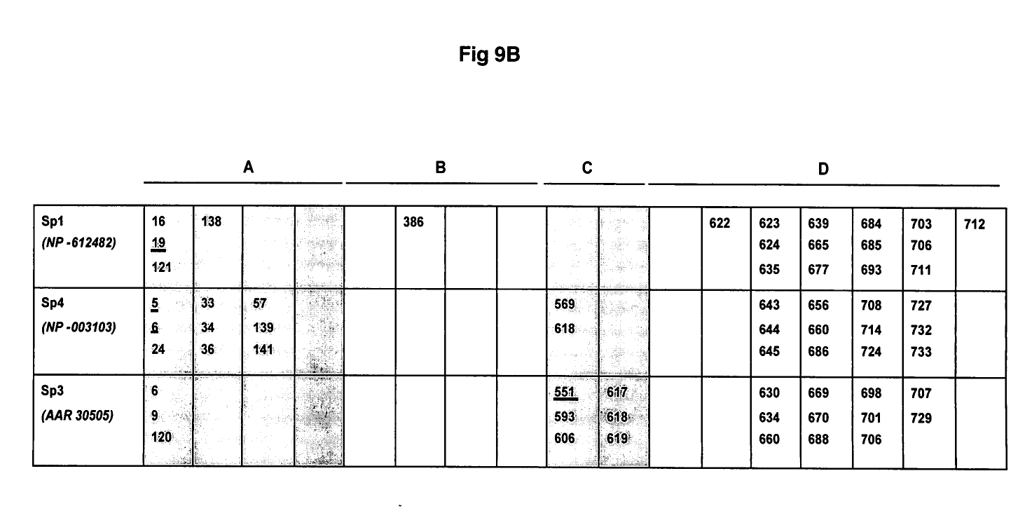

Gal4-Spx-y construct for Sp3 is generated using similar cloning technique also used for Sp1 and Sp4 (FIG. 9A-9B and FIG. 10). Plasmids expressing the Gal4 DNA binding domain (amino acids 1-147) fused to Sp3 wildtype (pGAL4 Sp3 wildtype) and to domain A (pGAL4 Sp3 A), are provided by Dr. Grace Gill (Harvard Medical School, Boston, Mass.). pGAL4 Sp1 B, CD, and D are prepared as follows: pGAL4-Sp3B (amino acid 263-542) fragment is PCR amplified using the primer set of 5′ TCC GTC GAC GCA ACA GCG TTT CTG CAG CTA CC 3′ (sense) and 5′ TAT CTA GAA TCA GCC TTG AAT TGG GTG CAC CTG 3′ (anti-sense); the fragment is digested with SalI and XbaI, and cloned into the pM construct. pGAL4-Sp3CD (amino acid 543-778) and pMGAL4-Sp3D (amino acid 622-778) is generated using PCR. The primer sets for pGAL4-Sp3CD are 5′ TCC GGA TCC GCC TGC CGT TGG CTA TAG CAA AT 3′ (sense) and 5′ GTA TGT CGA CAT CAG AAG CCA TTG CCA CTG ATA TT 3′ (antisense); and primer sets for pGAL4 Sp3D (amino acid 635-788) are 5′ TCC GGA TCC GCC TGC CGT TGG CTA TAG CAA AT 3′ (sense) and the same anti-sense primer above. The PCR products are digested with BamHI and SalI and cloned into pM (Gal4-DBD) constructs. Mutations of the selected lysine (K-one letter code) residue are generated by the site-directed mutagenesis method and verified by sequencing. Transfection of constructs into pancreatic cancer cells and preparation of nuclear extracts are described above.

### EXAMPLE 10

### Ubiquitinated Sp Proteins Immunoprecipitation

Panc1 and L3.6pl cells are seeded into 150-mm tissue culture plates in maintenance medium and allowed to grow to approximately 90% confluence. Cells are treated with DMSO, 50 μM of tolfenamic acid, or a more active non-steroidal anti-inflammatory drug for 7 hr. Whole cell extracts for each treatment group are obtained using radioimmunoprecipitation assay (RIPA) buffer (50 mM Tris-HCL, 1% NP-40, 0.25% Na-deoxycholate, 150 mM NaCl, 1 mM EDTA) with the addition of protease inhibitor cocktail. Duplicate aliquots of 500 μg are used for the experiments. Cell extracts are diluted in ice-cold phosphate buffered saline containing protease inhibitor cocktail to a final volume of 1 ml, followed by the addition of 30 μL of protein A/G PLUS-agarose beads (Santa Cruz). The reactions are rocked 4° C. for 3 hr, followed by centrifugation at 600 g at 4° C. for 5 min. An aliquot of 900 μL supernatant from each sample is transferred into a new EPPITUBE on ice. Rabbit polyclonal anti-Sp1 (1 μg), Sp4 (1 μg), Sp3 (1 μg), or normal rabbit IgG (1 μg) is added to the treatment groups, followed by addition of 30 μL of protein A/G PLUS-agarose beads. Samples are rocked at 4° C. for 12 hr, followed by centrifugation at 600 g at 4° C. for 5 min. The supernatant is removed by aspiration and immunoprecipitates are washed with two cycles of 1 ml of ice-cold RIPA buffer followed by 1 ml of ice-cold phosphate buffered saline using centrifugation at 600 g at 4° C. for 5 min. The agarose pellet is resuspended in 50 μL of loading buffer, boiled, and centrifuged. The supernatant is separated by SDS-10% PAGE, electrophoresed to polyvinylidene fluoride (PVDF) membrane. The PVDF membrane is probed with ubiquitin antibody (P4D1), then stripped and reprobed with Sp1 or Sp3 antibodies. The same membrane is stripped and reprobed with Sp4 antibody and visualized by enhanced chemiluminescence (ECL) as described (Abdelrahim et al, 2004).

### EXAMPLE 11

### SUMOylated Sp Proteins Immunoprecipitation

Panc1 and L3.6pl cells are seeded and treated as described for the ubiquitinated Sp protein immunoprecipitation experiment except that extract is pretreated with the cysteine protease inhibitor N-ethylmaleimide (NEM) to inhibit de-small ubiquitin-like modifier (SUMO)ylating enzyme. In addition, the PVDF membrane is probed with SUMO-1 antibody first, then stripped and re-probed with Sp1 or Sp3 antibodies. The same membrane is then stripped and re-probed with Sp4 antibody and visualized by enhanced chemiluminescence as described above.

### EXAMPLE 12

### Glycosylated Sp Proteins Immunoprecipitation

Panc1 and L3.6pl cells are seeded and treated as described for in the ubiquitinated Sp protein immunoprecipitation experiments (Example 10), except that the PVDF membrane is probed with the monosaccharide O-GlcNAc-specific RL-2 antibody first, stripped and reprobed with Sp1 or Sp3 antibodies. The same membrane is stripped and reprobed with Sp4 antibody and visualized by enhanced chemiluminescence, as described above.

### EXAMPLE 13

### Identification of Non-Steroidal Anti-Inflammatory Drugs that Downregulate Sp1, Sp3 and Sp4 Expression in Pancreatic Cancer Cells

RNA interference studies have reported that vascular endothelial growth factor is regulated by Sp1, Sp3 and Sp4 proteins in pancreatic cancer cells and celecoxib decreased Sp1 and VEGF expression in both in vitro and in vivo models (Abdelrahim et al, 2005a; Bouwman et al, 2002). Most studies on COX-1/2 inhibitors have shown growth inhibition at concentrations between 25-100 μM. Table 1 summarizes the list of individual non-steroidal anti-inflammatory drugs.

TABLE 1
Summary of non-steroidal anti-inflammatory drugs

| CLASS | COMPOUNDS |
|---|---|
| Salicylates | Aspirin, diflunisal, fendosal |
| Acetic acids | Indomethacin, acemetacin, anmetacin, sulindac, |
| | tolmetin, zomepirac, diclofenac, fenclofenac, |
| | isoxepac |
| Propionic acids | Ibuprofen, naproxen, betoprofen, fenoprofen, |
| | flurbiprofen, indoprofen, pirprofen, carprofen, |
| | fenbufen |
| Fenamates | Flufenamic acid, tolfenamic acid, mefenamic |
| | acid, meclofenamate, clonixin, flunixin, |
| | aceclofenac, niflumic acid |
| Pyrazoles | Febrazine, phenylbutazone, apazone, |
| | trimethazone, mofebutazone, bebuzone, |
| | suxibuxone |
| Osicams | Ampiroxicam, piroxicam, isoxicam, fenoxicam |

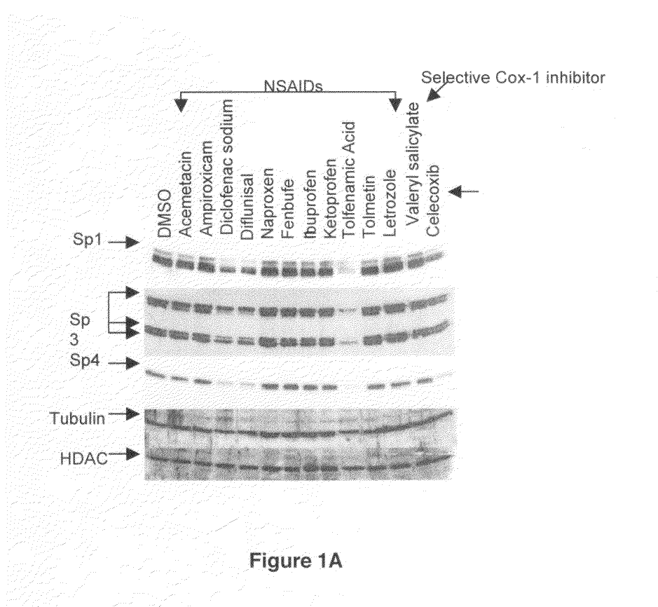

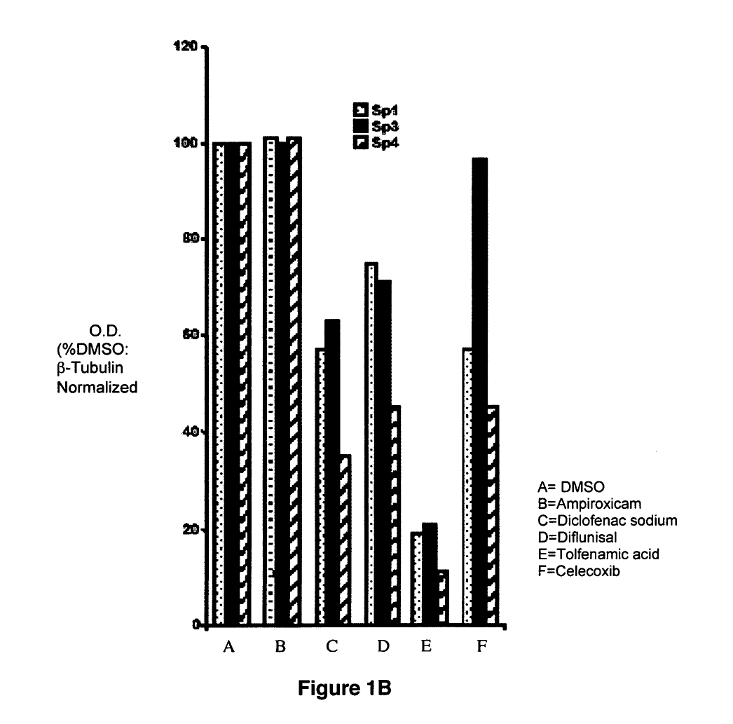

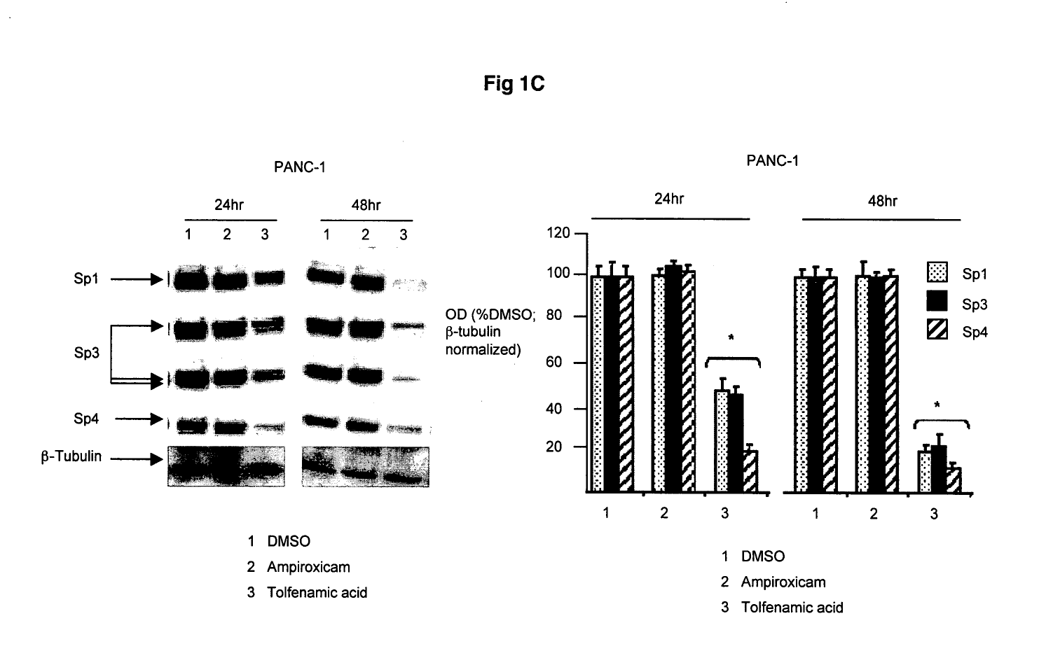

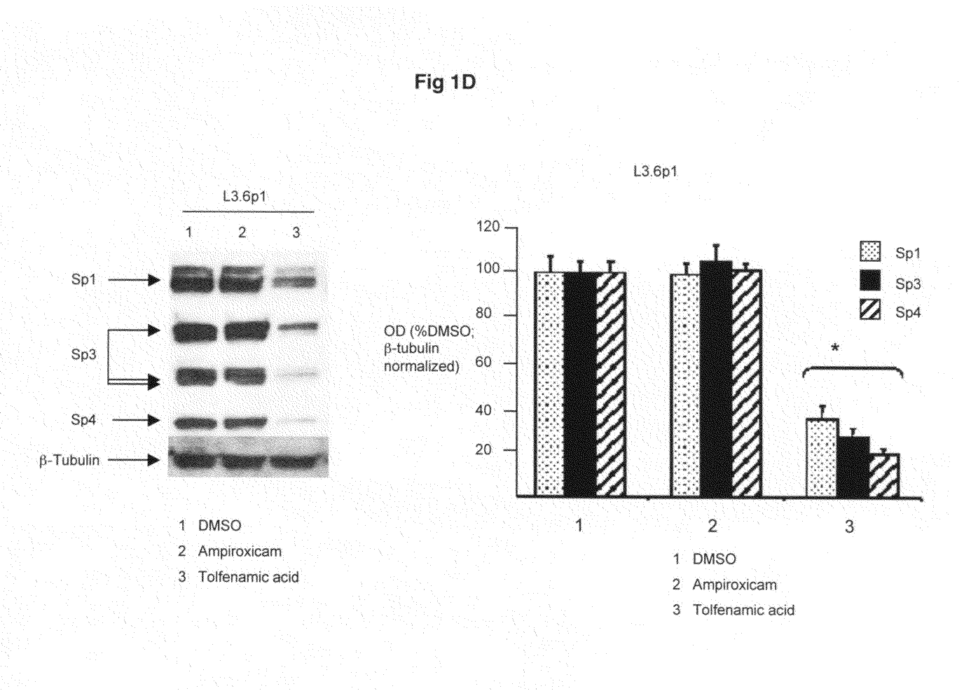

Hence, the present invention screened for the effects of 50 and 100 μM concentrations of different structural classes of non-steroidal anti-inflammatory drugs, celecoxib (COX-2 inhibitor) and valeryl salicylate (COX-1 inhibitor) on Sp1, Sp3, and Sp4 protein expression in Panc-1 cells after treatment for 48 hr. For the concentration of 50 μM (FIG. 1A), it was observed that celecoxib decreased the levels of Sp1 and Sp4 as previously described for colon cancer cells (Baker et al, 2002), whereas treatment with acemetacin, ampiroxicam, naproxen, fenbuten, ibuprofen, tolmetin, letrozole, or valeryl salicylate had no effect on the levels of these proteins. In distinct contrast, the diphenyl and diphenylamine-derived carboxylic acid compounds decreased expression of Sp1, Sp3, and Sp4 proteins in Panc-1 cells and their order of potency was tolfenamic acid>diclofenac˜diflunisal (FIG. 1B). Similar results were obtained using 100 μM concentration of non-steroidal anti-inflammatory drugs.

Additionally, 50 μM tolfenamic acid induced a time-dependent decrease in Sp1, Sp3, and Sp4 proteins in Panc-1 cells (FIG. 1C) with greater than 80% decrease in levels of all three proteins after treatment for 48 hr. In distinct contrast, treatment with ampiroxicam (negative control) did not affect Sp protein levels compared to those in solvent (DMSO)-treated cells. The comparative effects of 50 μM tolfenamic acid and ampiroxicam on Sp protein expression in the highly metastatic L3.6pl pancreatic cancer cell line (FIG. 1D) were similar to those observed in Panc-1 cells (FIG. 1C) and greater than 65% decrease of Sp1, Sp3 and Sp4 proteins was observed after treatment for 48 hr. Both dose (25-150 μM)—and time (12-76 hr)—dependent decreases in Sp1, Sp3, and Sp4 in pancreatic cancer cell lines, and intracellular decreases was confirmed by immunocytochemistry. Nuclear extracts are incubate with a [³²P]GC (consensus GC-rich oligonucleotide and analyzed in gel mobility shift assays (±Sp antibodies) as described (Abdelrahim et al, 2004; Wei et al, 2004).

### EXAMPLE 14

### Tolfenamic Acid Decreased Transactivation in Pancreatic Cancer Cells Transfected with VEGF Promoter Constructs

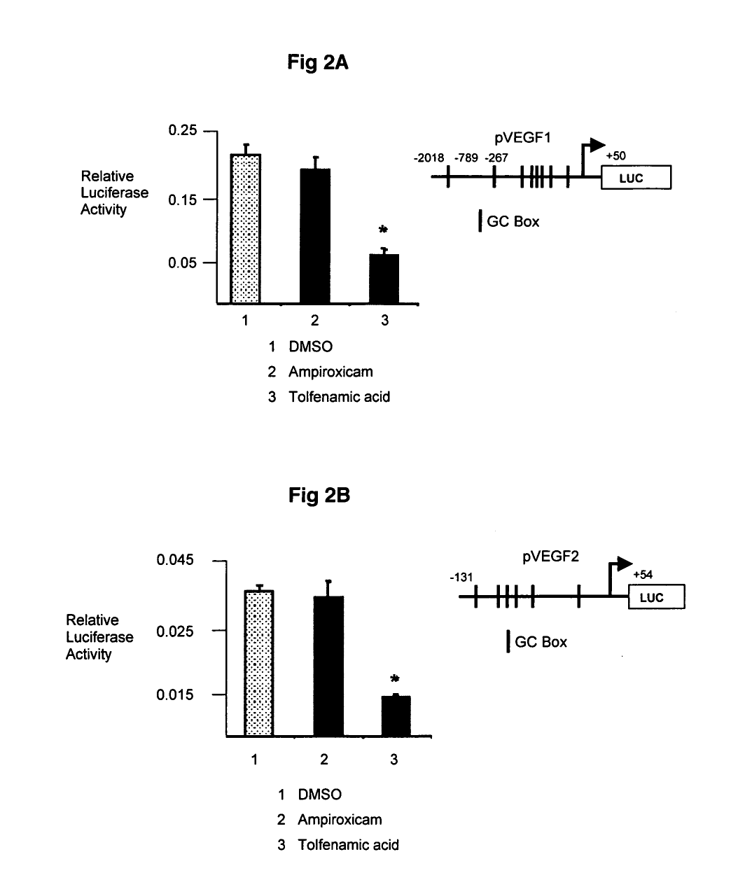

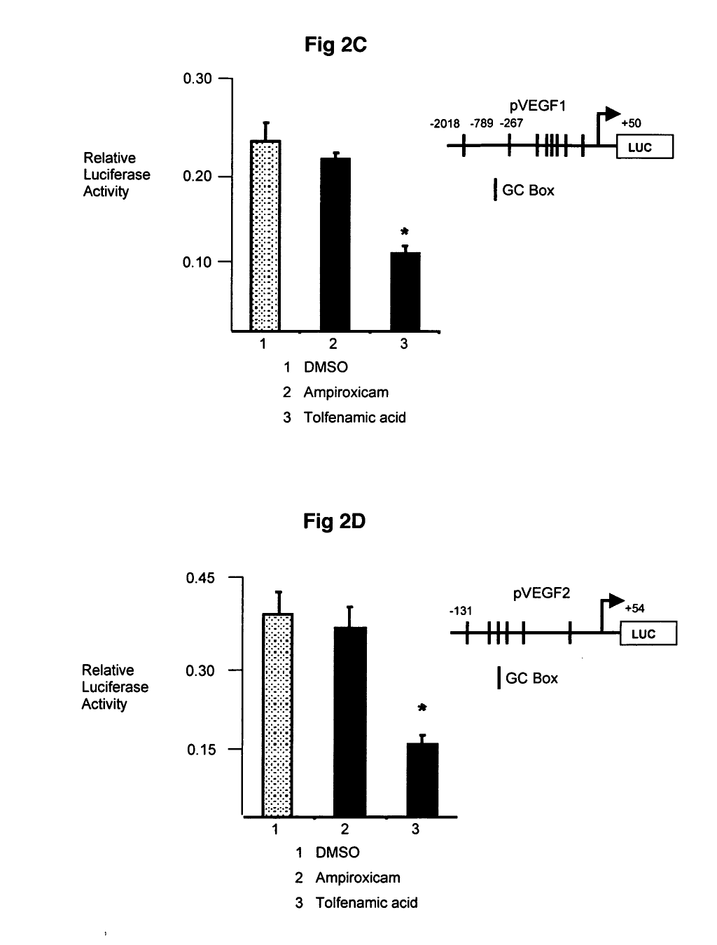

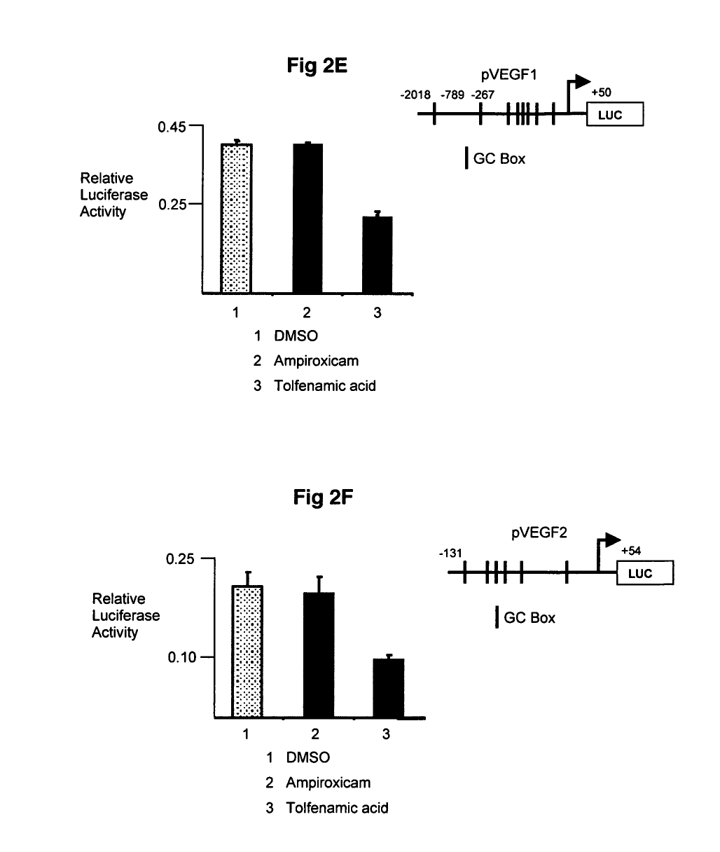

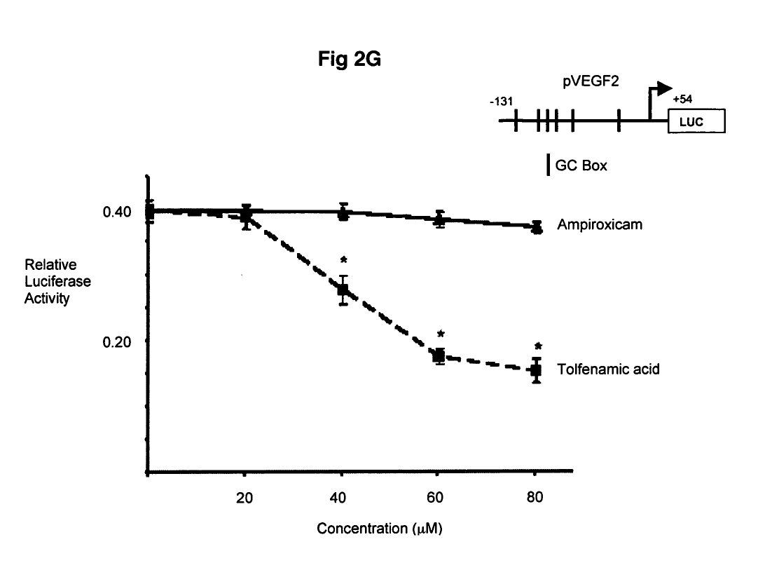

Previous studies have shown COX-2 inhibitors or small interfering RNAs for Sp proteins decreased vascular endothelial growth factor expression in colon and pancreatic cells (Abdelrahim et al, 2005; Baker et al, 2002; Masferrer et al, 2000; Bouwman et al, 2002). The effects of DMSO, 50 μM tolfenamic acid and 50 μM ampiroxicam on vascular endothelial growth factor expression were initially investigated in Panc-1 cells transfected with the pVEGF1 and pVEGF2 constructs, which contain −2018 to +50 and −131 to +54 VEGF promoter inserts, respectively (FIGS. 2A and 2B). Tolfenamic acid, but not ampiroxicam, significantly decreased activities in cells transfected with both constructs.

In a parallel series of experiments in Panc-1 cells, it was shown that diclofenac sodium which induces Sp protein degradation (FIG. 1A) also decreased transactivation in Panc-1 cells transfected with pVEGF1 (FIG. 2C) and pVEGF2 constructs (FIG. 2D). Decreased luciferase activity after treatment with tolfenamic acid and diclofenac sodium was consistent with previous studies that showed that activation of these constructs was dependent on interactions of Sp1, Sp3 and Sp4 proteins with proximal GC-rich motifs (Abdelrahim et al, 2005; Baker et al, 2002; Masferrer et al, 2000; Bouwman et al, 2002).

Additionally, a similar experiment was performed in L3.6pl cells (FIGS. 2E and 2F) and the results similar to those observed in Panc-1 cells were obtained. These data were consistent with the observed downregulation of Sp1, Sp3, and Sp4 protein in L3.6pl and Panc-1 cells treated with tolfenamic acid (FIG. 1). Furthermore, a concentration-dependent decrease in transactivation was observed in Panc-1 cells transfected with pVEGF2 and treated with 20-80 μM of tolfenamic acid (FIG. 2G), whereas ampiroxicam showed no effect. Similar results were obtained for the COX-1 inhibitor valeryl salicylate (data not shown).

### EXAMPLE 15

### Tolfenamic Acid Decreased Sp Protein Binding to GC-Rich VEGF Promoter Oligonucleotides

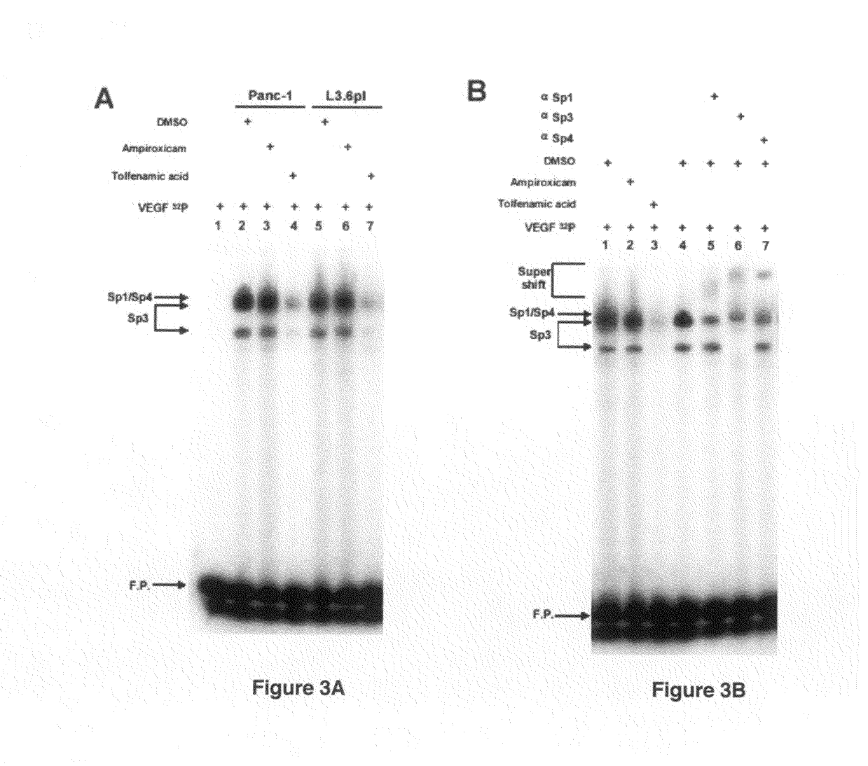

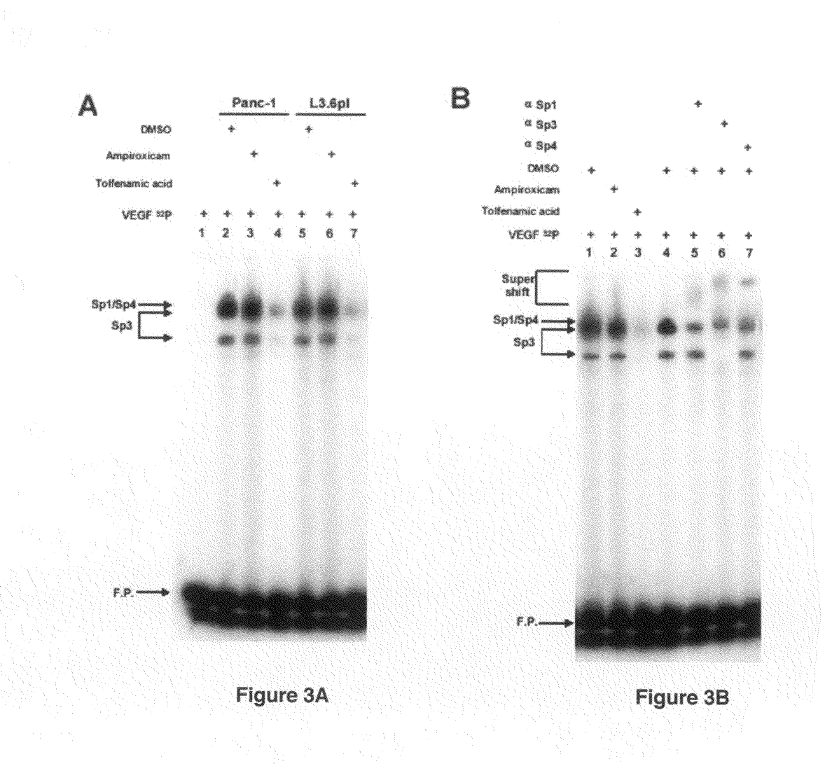

VEGF is regulated by Sp proteins binding to proximal GC-rich promoter sequences (Abdelrahim et al, 2005; Baker et al, 2002; Masferrer et al, 2000; Bouwman et al, 2002). Hence, the effects of tolfenamic acid and ampiroxicam on DNA binding was examined using mobility shift assay (FIGS. 3A and 3B). Panc1 or L3.6pl cells were treated with DMSO, 50 μM tolfenamic acid or 50 μM ampiroxicam for 48 hr. Nuclear extracts from these cells were isolated, incubated with VEGF³²P and analyzed in gel mobility shift assays.

The radiolabeled oligonucleotide contained a GC-rich region (−66 to −47) from the VEGF promoter that binds Sp proteins. Sp3 and Sp1/Sp4 (overlapping) retarded bands of similar intensity were observed after incubation with extracts from Panc-1 cells treated with DMSO or ampiroxicam (lanes 2 and 3) or comparable extracts from L3.6pl cells (lanes 5 and 6). In distinct contrast, these retarded bands were decreased using extracts from Panc-1 (lane 4) or L3.6pl (lane 7) cells treated with tolfenamic acid. Retarded bands were not observed using the VEGF ³²P alone (lane 1).

Additionally, results in FIG. 3B also illustrate the pattern of binding with extracts from cells treated with DMSO, ampiroxicam and tolfenamic acid (lanes 1-3, respectively). The retarded bands formed in extracts from solvent (DMSO)-treated cells (lane 4) exhibited supershifted bands after coincubation with antibodies for Sp1 (lane 5), SP3 (lane 6), and Sp4 (lane 7). The supershifted complexes are indicated by arrows. The results discussed herein demonstrated that tolfenamic acid decreased Sp1/Sp3/Sp4 binding to GC-richoligonucleotides identical with the −66 to −47 region of the VEGF promoter and this was consistent with decreased transactivation observed in transient transfection studies.

### EXAMPLE 16

### Non-Steroidal Anti-Inflammatory Drug-Induced Anti-Angiogenic Activity

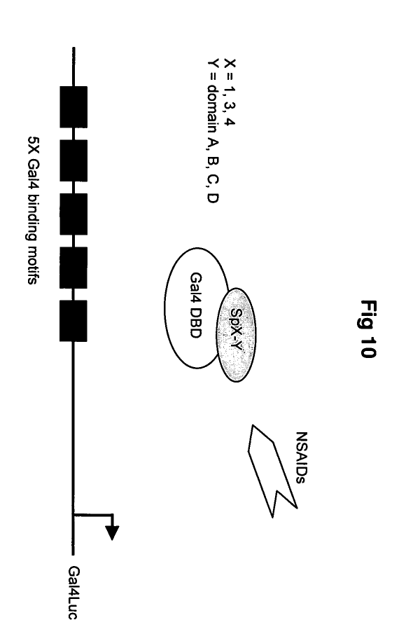

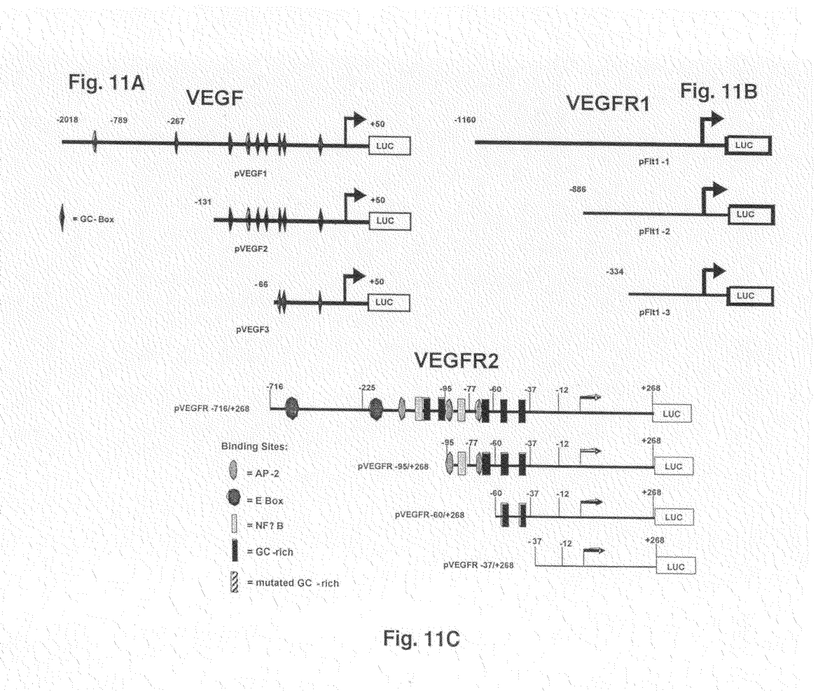

Using active non-steroidal anti-inflammatory drugs, the time- and dose-dependent decrease in vascular endothelial growth factor, VEGFR2 and VEGFR1 mRNAs/proteins is determined by western blot, RT-PCR, and immunocytochemistry to confirm down regulation of these angiogenic factors. Non-steroidal anti-inflammatory drug-induced decreases in VEGF/VEGFR1/VEGFR2 is also be confirmed in transient transfection studies using constructs containing promoter inserts (FIG. 11). These results correlate a critical response associated with down regulation of Sp proteins, namely down regulation of angiogenic factors regulated by Sp transcription factors.

Confirmation that tolfenamic acid decreased VEGFR1 expression in Panc-1 and L3.6pl cells was determined by immunohistochemical analysis (FIG. 15). In Panc-1 cells, VEGFR1 immunostaining was observed in cells treated with DMSO or ampiroxicam, whereas tolfenamic acid decreased VEGFR1 staining. VEGFR1 expression was lower in L3.6pl cells, however, the pattern of treatment related effects were comparable in both Panc-1 and L3.6pl cells.

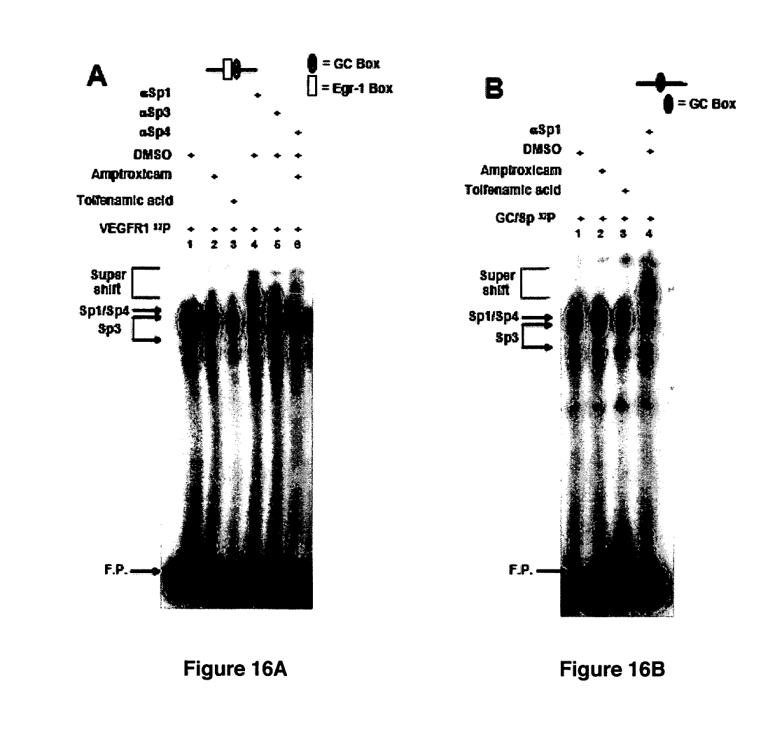

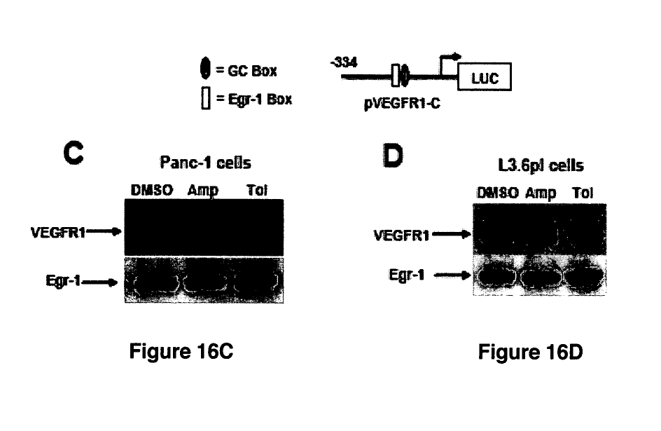

The proximal region of the VEGFR1 promoter contains GC-rich and Egr-1 sites. The effects of tolfenamic acid on VEGFR1 expression through degradation of Sp proteins cannot exclude a role for Egr-1 in this response. FIGS. 16A and 16B compare the gel mobility shift and antibody supershift patterns of nuclear extracts from Panc-1 cells bound to the proximal region of the VEGFR1 promoter containing both GC-rich and Egr-1 sites (VEGFR1³²P) or GC-rich sites alone (GC/SP³²P). Extracts from cells treated with DMSO, ampiroxicam and tolfenamic acid gave similar Sp1, Sp3, and Sp4-DNA retarded bands using both radiolabeled oligonucleotides (FIGS. 16A and 16B, lanes 1-3), however, the retarded band intensity was markedly decreased using extracts from cells treated with tolfenamic acid. Antibodies against Sp1 (FIGS. 16A and 16B, lane 4) supershifted the large Sp1-DNA retarded band and both Sp3 and Sp4 antibodies also induced formation of supershifted complexes (FIG. 16A, lanes 5 and 6). These results show that the presence or absence of the Egr-1 site did not affect Sp protein interactions with the VEGFR1 promoter. Moreover, western blot analysis of whole cell lysates from Panc-1 and L3.6pl cells after treatment with DMSO, ampiroxicam, or tolfenamic acid, showed only the latter compound decreased VEGFR1 protein levels, whereas Egr-1 protein was unchanged in all treatment groups (FIGS. 16C and 16D).

Since Sp proteins regulate expression of VEGFR1, the effects of tolfenamic acid on luciferase activity were further investigated in cells transfected with pVEGFRlA, pVEGFR1B and pVEGFR1C, and on VEGFR1 mRNA levels. Tolfenamic acid, but not DMSO or ampiroxicam, decreased luciferase activity in Panc-1 cells transfected with pVEGFR1A, pVEGFR1B and pVEGFR1C (FIGS. 17A-17C) and tolfenamic acid also decreased VEGFR1 mRNA levels in Panc-1 and L3.6pl cells (FIG. 17D).

Previous studies have reported that activation of VEGFR1 by VEGF-B in pancreatic cancer cells results in enhanced phosphorylation of MAPK and increased cell migration and invasion. The role of Sp proteins in mediating these responses was therefore investigated by determining the effects of tolfenamic acid on activation of MAPK by VEGF-B. FIGS. 18A and 18B illustrate that after treatment of Panc-1 and L3.6pl cells, respectively, with VEGF-B for 5 or 10 min, there was increased phosphorylation of MAPK1/2 in cells pretreated with DMSO or 50 μM ampiroxicam for 36 hr, and VEGFR1 and total MAPK1/2 levels were unchanged. In contrast, pretreatment with 50 μM tolfenamic acid decreased VEGFR1 expression, and this was paralleled by decreased phospho-MARK1/2, whereas total MAPK protein levels were not affected. Thus, inhibition of VEGF-B/VEGFR1 signaling by tolfenamic acid was related to decreased VEGFR1 through degradation of Sp proteins and this inhibitory response was similar to that observed in a previous study using neutralizing VEGFR1 antibodies. The importance of Sp protein in VEGFR1-dependent Panc-1 cell migration was determined using a cell migration assay on collagen IV-coated plates. The results show that VEGF-B induces Panc-1 cell migration in all treatment groups (FIG. 18C); however, in cells treated with DMSO (set at 100%), 50 μM ampiroxicam, or 50 μM tolfenamic acid cell migration which was observed in the absence of VEGF-B was only significantly inhibited by tolfenamic acid (FIG. 18C). VEGF-B enhanced cell migration in this assay, and in cells co-treated with VEGF-B plus tolfenamic acid, the latter compound significantly inhibited VEGF-B-induced cell migration.

Previous studies using an orthotopic model for pancreatic cancer (using L3.6pl cells) showed that tolfenamic acid (50 mg/kg) but not gemcitabine decreased Sp proteins, tumor growth, and angiogenesis, and tumor tissue from these animals was also stained for VEGFR1 (FIG. 18D). The results show decreased VEGFR1 expression only in tumors from tolfenamic acid-treated mice. These results demonstrate that Sp proteins regulate VEGFR1-mediated responses including cell migration in pancreatic cancer cells indicating that agents such as tolfenamic acid that target Sp proteins (for degradation) are an important new class of mechanism-based anti-angiogenic compounds that decrease Sp-dependent expression of VEGF, VEGFR2 and VEGFR1.

### EXAMPLE 17

### Role of Proteasome Pathway in Sp Protein Degradation

The role of the proteasome pathway in mediating degradation of Sp proteins is investigated. Posttranslational modification of Sp proteins are summarized in Table 2 (phosphorylation, sumoylation and ubiquitination) can markedly affect their expression/activity in cells, and the effects of tolfenamic acid on these modifications of Sp proteins will be investigated in Panc1 and L3.6pl cells.

TABLE 2
Post-translational modification of Sp proteins.*

| | | | Sp4 |
|---|---|---|---|
| Post-translational | Sp1 | Sp3 | (Amino acid and |
| modification | (Amino acid and Domain) | (Amino acid and Domain) | Domain) |
| Glycosylation | Ser/Thr residues | Not determined | Not determined |
| | Activation domain | | |
| Phosphorylation | Ser/Thr residues, | Not determined | Ser/Thr |
| | some are also targeted | | residues |
| | by glycosylation | | Activation |
| | Activation domain | | domain and |
| | and DNA-binding | | DNA-binding |
| | domain | | domain |
| Acetylation | Lysine | Inhibitory | Not determined |
| | DNA-binding domain | domain | |
| | | (IKEE motif) | |
| | | DNA binding | |
| | | domain | |
| Sumoylation | Not determined | Inhibitory | Not determined |
| | | domain | |
| | | (IKEE motif- | |
| | | Lysine 551) | |
| Ubiquitination | Lysine | Not detected | Lysine |
| | N-terminal and other | | N-terminal and |
| | domains | | other domains |

*The various domains of Sp proteins

Tolfenamic acid and/or two active non-steroidal anti-inflammatory drugs that target Sp proteins for degradation are investigated to understand the mechanisms of Sp1, Sp3, and Sp4 protein degradation, and the role of the proteasome pathway in this process. Pancreatic cancer cells are co-treated with non-steroidal anti-inflammatory drugs and several proteasome and proteasome/protease inhibitors for 36-48 hr, and Sp protein levels are analyzed by western blot analysis and immunohistochemistry.

Inhibition of Sp protein degradation by tolfenamic acid and related compounds is quantified relative to the control treatment (DMSO) and the structural protein (β-tubulin). The peptide aldehydes (such as N-acetyl-L-leucinyl-L-leucinal-L-norleucinal, LLnL, and N-carbobenzoxyl-L-leucinyl-L-leucinyl-L-norvalinal, MG115) are used and these peptides strongly inhibit peptidase activities associated with the proteasome complex (Rock et al, 1994). However, these peptide aldehydes can also inhibit the cysteine proteases found in lysosomes and calpains (Rock et al, 1994). Therefore, more selective proteasome inhibitors are used to definitively establish a major role for proteasomes in Sp protein degradation by non-steroidal anti-inflammatory drugs.

Lactacystin is a chemically distinct proteasome inhibitor that reversibly inhibits the post-acidic activity of purified proteasomes through formation of clasto-lactacystin β-lactone, which reacts with the N-terminal threonine of subunit X (Omura et al, 1991; Dick, 1996). Lactacystin and gliotoxin are used as proteasome inhibitors to confirm that Sp proteins are degraded by proteasomes.

### EXAMPLE 18

### Identification of Structural Domains that are Required for Sp1, Sp3, and Sp4 Protein Degradation by Tolfenamic Acid and Related Non-Steroidal Anti-Inflammatory Drugs

The proteasome complex recognizes its specific targets in several different pathways and these are related, in part, to modification of critical lysine residues with ubiquitin or ubiquitin-like peptides that are recognized by the proteasome complex (see below). FIG. 9A-9B summarizes the domain structures of Sp1, Sp3, and Sp4, and this contains activation domains (AD), inhibitory domains (ID), and the characteristic C-terminal zinc finger motifs required for DNA binding. The present invention has prepared Gal4-Sp1/Gal4-Sp3/Gal4-Sp4 constructs, and the truncated forms contain the C-terminal A, N-terminal D, and internal C and B domains of Sp1 and Sp3 fused to the yeast Gal4 DBD (amino acids 1-147). The corresponding cDNAs encoding various fragments of Sp1/Sp4 are generated by restriction enzyme digested and PCR amplification using high fidelity Pfu DNA polymerase and cloned into the Gal4 DBD vector. The resulting plasmids [Gal4/Sp3(A-D) fragments] are used along with the corresponding wild-type/mutant GAL4-Sp1/Gal4-Sp4 in studies designed to determine which domains of Sp proteins are targeted for degradation.

Plasmid constructs that express chimeric proteins containing the DBD of the yeast Gal4 protein fused to different domains of Sp proteins (Gal4-Sp) (FIG. 10) are transfected into Panc1 and L3.6pl cells in 6-well plates along with 500 ng reporter plasmid (Gal4-Luc) containing five tandem Gal4 response elements linked to bacterial luciferase. Cells are then treated with different concentrations of non-steroidal anti-inflammatory drugs (15-100 μM). Decreased responses are indicative of non-steroidal anti-inflammatory drugs-induced degradation of specific domains of Sp1, Sp3, and Sp4, and thereby identify specific domains susceptible to proteasome degradation. That decreased transactivation is linked to activation of the proteasome pathway by non-steroidal anti-inflammatory drugs is confirmed using the proteasome inhibitors that will block the decreased transactivation in transfected cells.

Previous studies have demonstrated that non-steroidal anti-inflammatory drugs (COX-2 inhibitors) induce proteasome-dependent degradation of Sp1 and Sp4 in colon cancer cells, and this is associated with increased ubiquitination of both proteins (Abdelrahim et al, 2005). The present invention uses pancreatic cancer cells, which are treated with different concentrations of non-steroidal anti-inflammatory drugs for 24 hr, and whole cell lysates are immunoprecipitated with IgG or antibodies to Sp1, Sp3, or Sp4 and analyzed by western blot for Sp proteins and fubiquitinated proteins. These studies confirm that non-steroidal anti-inflammatory drugs induce ubiquitination of wild-type (endogenous) Sp1 and Sp4 in pancreatic cancer cells as previously observed in colon cancer cells (Abdelrahim et al, 2005). These same approaches are used for the wild-type/mutant Gal4-Sp constructs in which the chimeric proteins are immunoprecipitated with the Gal4 antibodies and analyzed by SDS-PAGE/western blot with ubiquitin antibodies.

In studies of colon cancer cells, COX-2 inhibitors did not induce degradation (or ubiquitination) of Sp3, and in the present invention, ubiquitination of Sp3 is also investigated. However, Sp3 is expressed as four isoforms that become post-translationally modified by SUMOylation (Wei et al, 2004) in which Sp3 is conjugated with small ubiquitin-related modifier (SUMO) (Johnson and Gupta AA, 2001; Tatham et al, 2001; Pichler et al, 2002; Rodriguez et al, 2001; Sapetschnig et al, 2002; Bernier-Villamor et al, 2002). A consensus SUMO acceptor site consisting of the sequence ψKXE has been identified where ψ is a large hydrophobic amino acid and K (lysine) is the site of SUMO conjugation and SUMO modification of Sp3 is specific at lysine residue 551. Therefore, the same approach is used for detecting SUMOylation of Sp3, Sp 1, and Sp4 by immunoprecipitation/SDS-PAGE and western blot analysis as described for ubiquitination of wild-type/variant Sp1 and Gal4-Sp proteins. FIG. 10 also summarizes the lysine sites in Sp proteins including lysine residues at amino acids 16 and 19 (for Sp1) and 5 and 6 (for Sp4) and, for Sp1, these amino acids are known to be important for proteasome degradation. Lysine residues within domains of Sp1, Sp3, and Sp4 that undergo non-steroidal anti-inflammatory drugs-induced proteasome degradation are mutated to alanines and their degradation is investigated in pancreatic cancer cells as described for the wild-type constructs. The lysine mutation studies confirms the role of specific lysines in Sp proteins in mediating their non-steroidal anti-inflammatory drug-induced degradation.

### EXAMPLE 19

### Post-Translational Modifications of Sp Proteins Induced by Tolfenamic Acid

Sp proteins are also subjected to other post-transcriptional modifications (Bruan et al, 2001) including phosphorylation, acetylation, and glycosylation (Black et al, 2001; Hart, 1997; Jackson and Tjian, 1988; Jackson et al, 1990). Phosphorylation and acetylation of Sp proteins can modify their activities as transcription factors but does not affect proteins expression (Chu and Ferro, 2005; Ryu et al, 2003; Suzuki et al, 2000; Huang et al, 2005; Braun et al, 2001). In contrast, glycosylation has been associated with protein stability and susceptibility to degradation (Chou et al, 1995; Kelly et al, 1993; Reason et al, 1992). This modification involves the covalent linkage of the monosaccharide O-GlcNAc to serine or threonine residues. The Sp1 protein was the first transcription factor known to contain this modification (Reason et al, 1992). Hypoglycosylated Sp1 is more susceptible to proteasome-dependent degradation and each molecule of Sp1 contains an average of eight O-Glc-NAc modifications (Reason et al, 1992), and one of these sites is in the transcriptional activation domain of the molecule. To determine if the glycosylation states of Sp1, Sp3, and Sp4 are affected by treatment with non-steroidal anti-inflammatory drugs, glucosamine, which is used primarily as a substrate for protein glycosylation, is used to test whether it can block Sp protein hypoglycosylation and degradation by non-steroidal anti-inflammatory drugs. Glucosamine treatment produced higher molecular weight hyperglycoslated forms of Sp1 protein and blocked its degradation under glucose starvation condition (Reason et al, 1992). Panc1 and L3.6pl cells treated with 5 mM glucosamine and/or different concentrations of non-steroidal anti-inflammatory drugs for 24 and 48 hr, are analyzed for Sp1, Sp3, and Sp4 proteins levels by western blotting. To investigate the glycosylation state of Sp1/Sp3/Sp4 proteins after non-steroidal anti-inflammatory drug treatment, cells are co-treated with 3 μM lactacystin to prevent loss of Sp proteins by non-steroidal anti-inflammatory drugs, then Sp proteins are immunoprecipitated by Sp specific antibodies and analyzed for glycosylation by western blot using RL-2 epitope GlcNAc-specific antibody.

### EXAMPLE 20

### Tolfenamic Acid Decreased Vascular Endothelial Growth Factor mRNA and Protein Expression and Activated Proteosome-Dependent Degradation of Sp1, Sp3, and Sp4

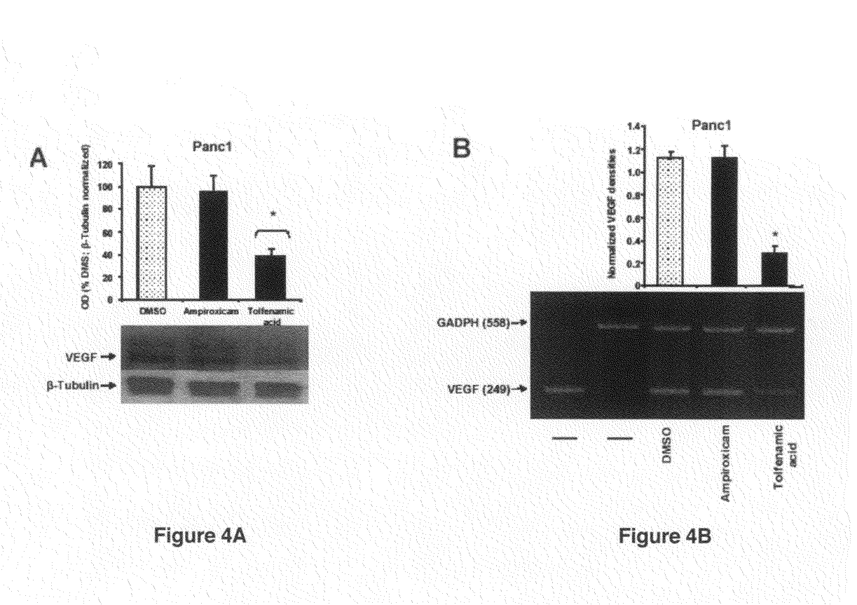

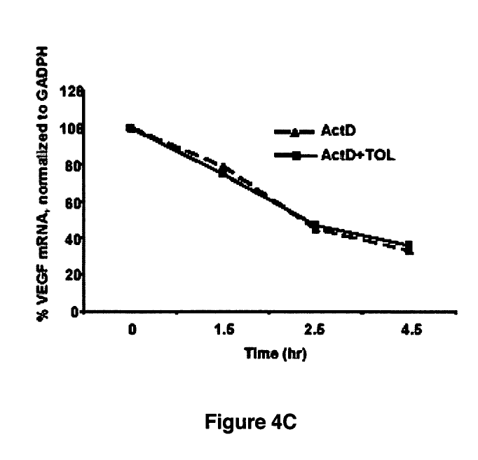

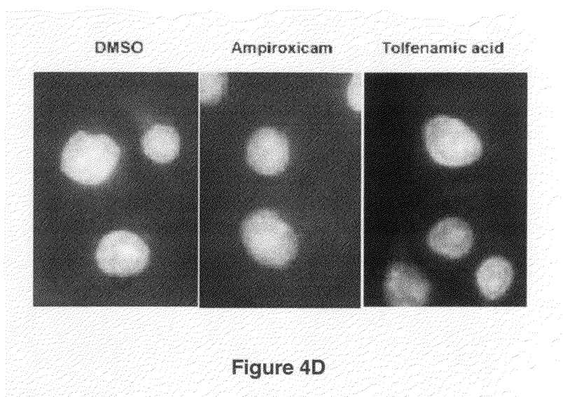

Panc-1 pancreatic cancer cells were treated with DMSO, 50 μM ampiroxicam or tolfenamic acid for 48 hr and VEGF protein levels (relative to β-tubulin) were analyzed (FIG. 4A). The results showed that tolfenamic acid decreased vascular endothelial growth factor expression by greater than 60% compared to solvent (DMSO) control. The effects of tolfenamic acid on vascular endothelial growth factor mRNA levels were also investigated in Panc-1 cells by RT-PCR and compared to levels in solvent (DMSO) and ampiroxicam-treated cells.

Tolfenamic acid treatment for 24 hours decreased vascular endothelial growth factor mRNA levels by greater than 60% (compared to DMSO) (FIG. 4B). However, this decrease was not due to decreased message stability since vascular endothelial growth factor mRNA levels decreased at similar rates in Panc-1 cells treated with actinomycin D alone, or in combination with 50 μM tolfenamic acid. In Panc-1 cells, vascular endothelial growth factor protein was secreted and immunostaining with vascular endothelial growth factor antibodies showed a significant lawn of extracellular vascular endothelial growth factor staining in cells treated with DMSO or ampiroxicam (FIG. 4D). In distinct contrast, after treatment of Panc-1 cells for 48 hr with 50 μM tolfenamic acid, the immunostaining of secreted vascular endothelial growth factor protein was almost non-detectable. These results clearly demonstrated that tolfenamic acid-dependent down regulation of Sp1, Sp3, and Sp4 protein expression resulted in decreased levels of vascular endothelial growth factor mRNA and protein which was consistent with the reported Sp-dependent regulation of vascular endothelial growth factor (Abdelrahim et al, 2005; Baker et al, 2002; Masferrer et al, 2000; Bouwman et al, 2002).

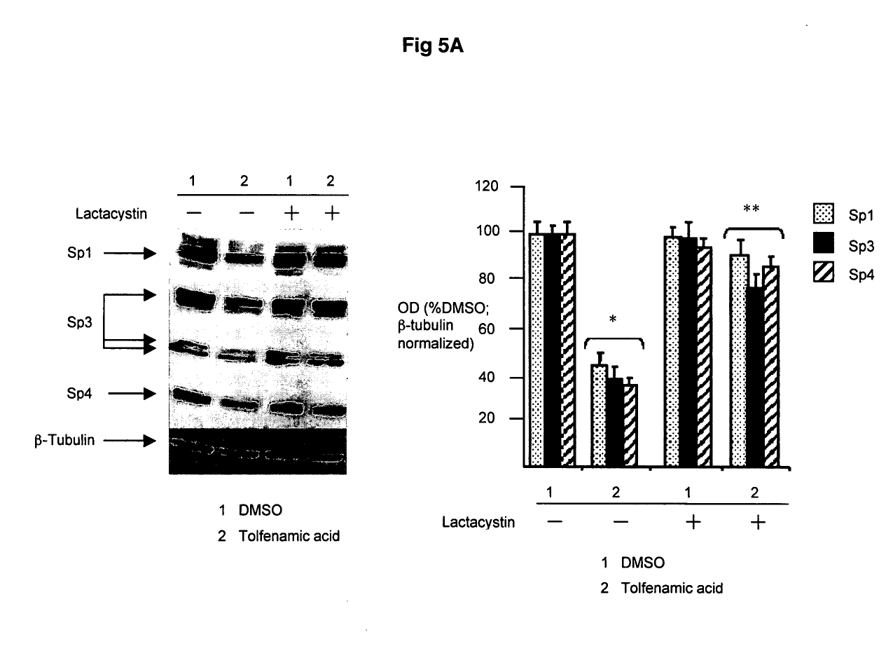

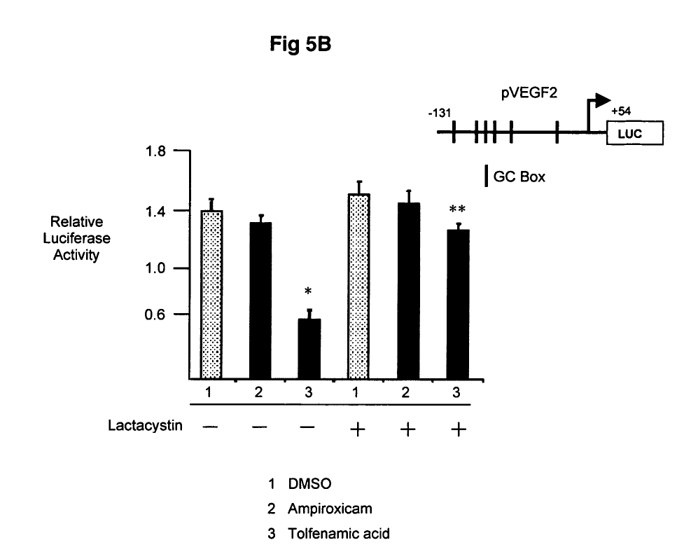

Furthermore, it was also reported that the COX-2 inhibitor, nimesulide, decreased levels of Sp1 and Sp4 (but not Sp3) in colon cancer cells through activation of the proteasome pathway, and this response was blocked by the proteasome inhibitor gliotoxin (Baker et al, 2002). The results in FIG. 5A demonstrated that tolfenamic acid decreased Sp1, Sp3, and Sp4 proteins and differed from nimesulfide (Baker et al, 2002), and celecoxib (FIG. 1A), which induced degradation of Sp1 and Sp4. but not Sp3. However, in Panc-1 cells co-treated with 50 μM tolfenamic acid plus 2 μM of proteosome inhibitor lactacystin, there was a significant inhibition of Sp1, Sp3, and Sp4 protein degradation. Thus, like COX-2 inhibitors (celecoxib and nimesulide), tolfenamic acid activated proteasome-dependent degradation of Sp1 and Sp4 in Panc-1 cells. Although non-steroidal anti-inflammatory drugs induced degradation of Sp3, COX-2 inhibitors had no effect on Sp3. Additionally, decreased transactivation in Panc-1 cells transfected with pVEGF2 and treated with tolfenamic acid was also blocked in cells co-treated with lactacystin (FIG. 5B) and similar results were obtained for gliotoxin. Ampiroxicam did not affect luciferase activity in presence or absence of lactacystin. These results also confirmed the role of Sp proteins as key mediators of vascular endothelial growth factor expression.

Treatment of SEG-1 and Bic-1 esophageal cancer cells with 25-100 μM tolfenamic acid decreased protein expression of Sp1, Sp3, and Sp4 (FIGS. 20A-20B), and decreased expression of Sp-dependent proteins vascular endothelial growth factor (VEGF), survivin, and c-Met (FIGS. 21A-21B), and induced apoptosis as shown by poly (ADP-ribose) polymerase (PARP) cleavage (FIG. 21A).

### EXAMPLE 21

### Tolfenamic Acid Inhibited Growth of Pancreatic Cancer Cells

Although it was reported that non-steroidal anti-inflammatory drugs and COX-2 inhibitors inhibited the growth of pancreatic cancer cells, the underlying mechanisms are not well understood. Sp proteins play a key role in regulating genes involved in cancer cell growth and angiogenesis, their role in pancreatic cancer cell growth was investigated in cells treated with tolfenamic acid and other non-steroidal anti-inflammatory drugs.

To determine the growth inhibitory effects of the active-non-steroidal anti-inflammatory drugs in pancreatic cancer cell cells, cell cycle phase distribution is determined by staining with propidium iodide and analysis by flow cytometry using the FACSCALIBUR® Immunocytometry Systems benchtop flow cytometer combined with a MACINTOSH® computer using CELLQUEST® software (Abdelrahim et al, 2004). Co-staining of the cells for direct incorporation of bromodeoxyuridine (BrdU) is carried out using direct immunofluorescence microscopy, anti-BrdU primary antibodies, and rhodamine-conjugated secondary antibodies.

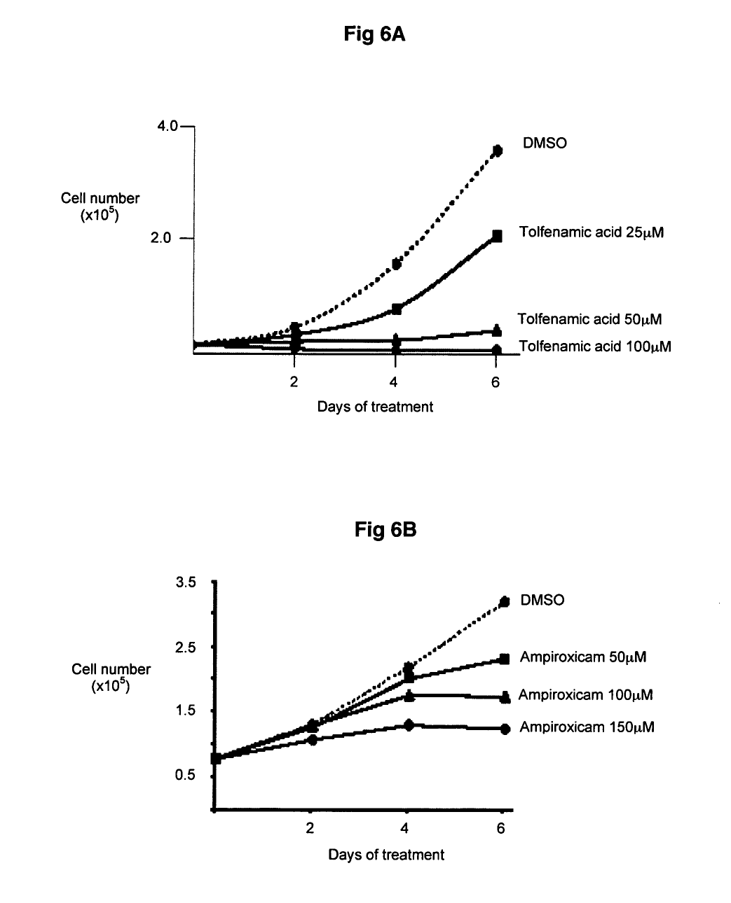

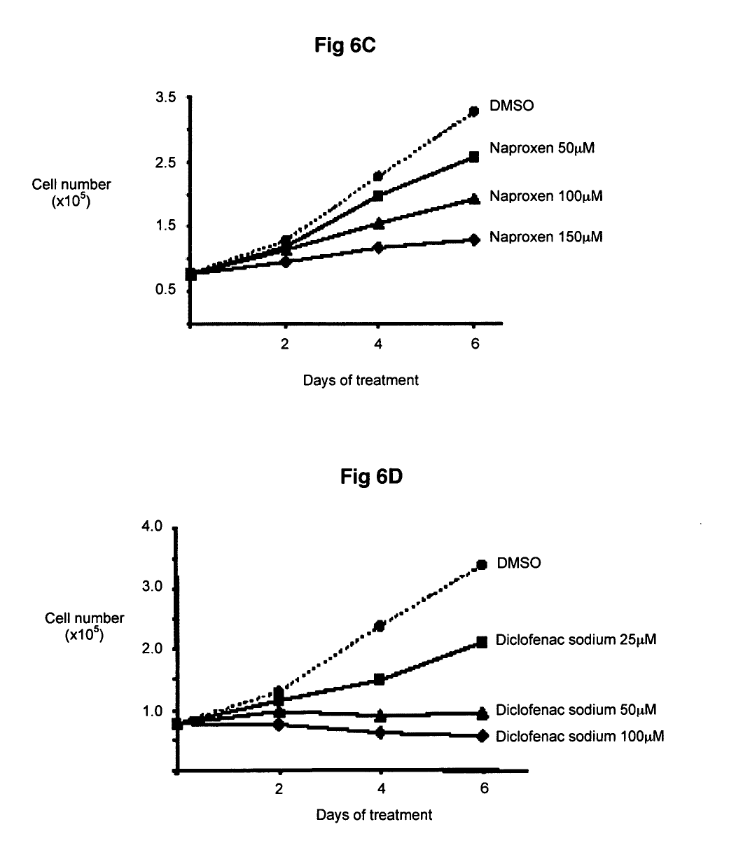

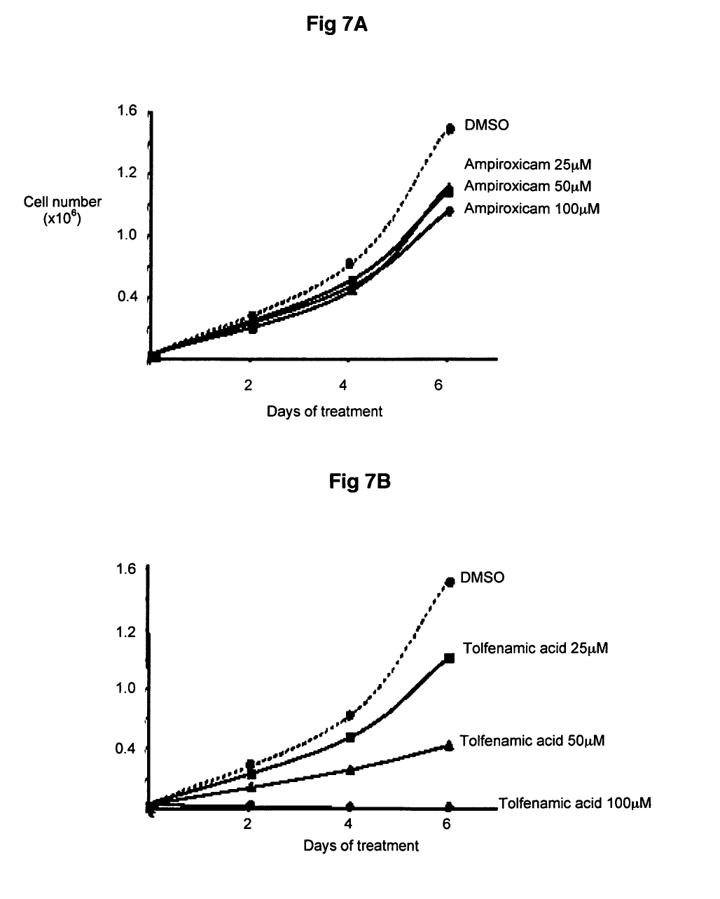

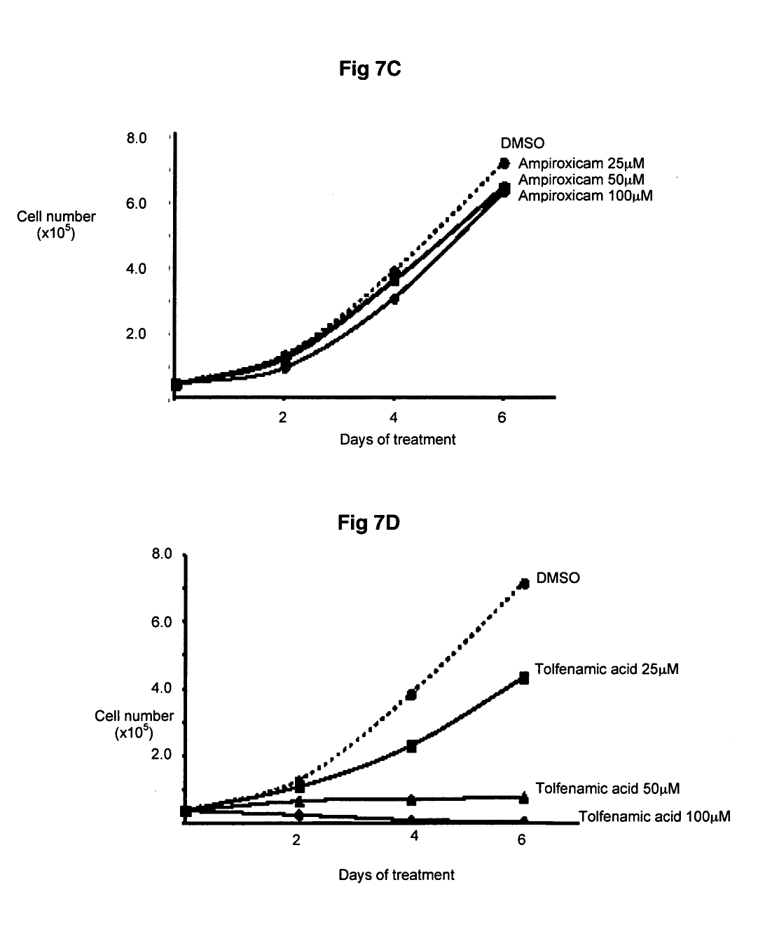

It was observed that ampiroxicam, naproxen, diclofenac sodium, and tolfenamic acid, decreased growth of Panc-1 pancreatic cancer cells and the latter two compounds also exhibited higher potencies in this assay (FIGS. 6A-6D). Since it was possible that the observed differences in the effects of 50 μM concentrations of non-steroidal anti-inflammatory drugs on Sp protein degradation (FIG. 1A) may be related in part to their relative growth inhibitory activities, the effects of tolfenamic acid, ampiroxicam, and naproxen on Sp protein expression was compared using concentrations that induced comparable inhibition of Panc-1 cell proliferation. Thus, Panc-1 cells were treated with 50 μM tolfenamic acid, 150 μM naproxen and 150 μM ampiroxicam for 48 hr and the expression of the proteins compared by western blot. It was observed that only tolfenamic acid induced protein degradation and poly (ADP-ribose) polymerase cleavage. These data supported the results of the initial screening assay (FIG. 1A) showing that only non-steroidal anti-inflammatory drugs containing the diphenyl/diphenylamine carboxylic acid structure induced Sp protein degradation. Using a similar range of concentrations in L3.6pl (FIGS. 7A and 7B) and Panc-28 (FIGS. 7C and 7D) cells, it was apparent that tolfenamic acid was also more potent as an inhibitor of pancreatic cell growth than ampiroxicam. These results suggested that the growth inhibitory effects of tolfenamic acid in pancreatic cells were associated in part, with degradation of Sp proteins.

### EXAMPLE 22

### Modulation of p27 and Other cdk1 Proteins/Genes

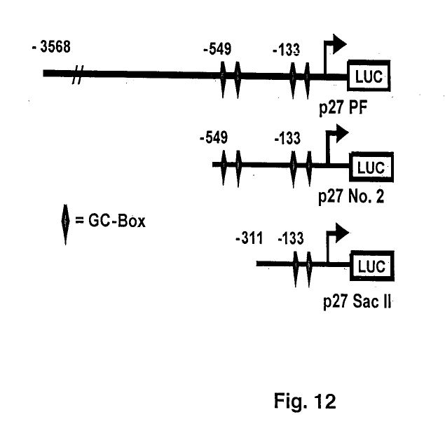

Recent studies show that Sp3 protein suppresses the cdk1, p27 and Sp3 knockdown by RNA interference, induces p27 expression, and inhibits G1 to S phase progression (Abdelrahim et al, 2004). Three active non-steroidal anti-inflammatory drugs are used and cells are treated as described above. Expression of p27 protein/mRNA is determined, and the effects on cell proliferation and the percentage distribution of cells in G₀G₁ S and G₂/M phases of the cell cycle is determined by FACS analysis as described (Abdelrahim et al, 2004). In addition, the effects of non-steroidal anti-inflammatory drugs on p27 promoter expression is examined using a series of GC-rich constructs (FIG. 12).

### EXAMPLE 23

### Orthotopic Implantation of L3.6pl Cells and Treatment with Non-Steroidal Anti-Inflammatory Drugs, Gemcitabine and Their Combination

At least three active non-steroidal anti-inflammatory drugs are administered at doses of 10, 25, and 50 mg/kg day, and gemcitabine is used at doses of 50, 75, and 100 mg/kg/day. In combination treatments, a minimally effective dose of gemcitabine in combination with at least two doses of the non-steroidal anti-inflammatory drugs is used to determine potential interactive effects on tumor growth liver metastasis and histopathology.

Male athymic nude mice (NCI-nu) are purchased from the Animal Production Area of the National Cancer Institute Frederick Cancer Research and Development Center, housed and maintained under specific pathogen-free conditions in accordance with current regulations and standards of the United States Department of Agriculture. Injection of L3.6pl cells (with greater than 90% viability is performed as previously described (Bruns et al, 1999). Seven days after implantation of tumor cells into the pancreas of each mouse, five mice are killed to confirm the presence of tumor lesions.

Mice (10 animals per treatment group) are randomized to receive one of the following treatments: (1) thrice weekly oral administrations of vehicle solution which will serve as a control group; (2) thrice weekly oral administrations of at least three non-steroidal anti-inflammatory drugs at doses of 10, 25, and 50 mg/kg/day; (3) twice weekly intraperitoneal injections of 50, 75, and 100 mg/kg/day gemcitabine alone; and (4) combinations of gemcitabine and non-steroidal anti-inflammatory drugs. Mice are sacrificed on day 35 and body weights measured. Primary tumors in the pancreas are excised, measured, weighed. For immunohistochemistry (IHC) and hematoxylin and eosin (H&E) staining procedures, one part of the tumor tissue is fixed in formalin and embedded in paraffin, and another part is embedded in TISSUE-TEK® O.C.T. compound, rapidly frozen in liquid nitrogen, and stored at −70° C. Visible liver metastases are counted with the aid of a dissecting microscope, and the tissues processed for hematoxylin and eosin staining.

### EXAMPLE 24

### Immunohistochemical Determination of Vascular Endothelial Growth Factor Sp Proteins and VEGFR2

Paraffin-embedded tissues are used for identification of Sp proteins, VEGFR2p27 and vascular endothelial growth factor. Tissue sections (4-6 μM thick) are mounted on positively charged SUPERFROST slides (Fischer Scientific, Co, Houston, Tex.) and dried overnight. Sections are deparaffinized in xylene and treated with a graded series of alcohol [100, 95, and 80% ethanol (v/v) in double distilled H₂O] and rehydrated in phosphate buffered saline (pH 7.5). Tissues are treated with pepsin (Biomeda) for 15 min at 37° C. and washed with phosphate buffered saline. A positive reaction is visualized by incubating the slides with stable 3,3′-diaminobenzidine for 10-20 min. Sections are rinsed with distilled water, counterstained with Gill's hematoxylin for 1 min, and mounted with UNIVERSAL MOUNT (Research Genetics). Control samples incubated with secondary antibody alone are negative. Tumor sample lysates are also examined by western blot analysis for the same proteins.

### EXAMPLE 25

### Sp Protein Knockdown by RNA Interference In Vivo

Recent studies show that siRNAs and derivatized siRNA can be injected into mice and efficient knockdown of target proteins observed for extended time periods. The present invention uses the SISTABLE™ (Dharmacon) siRNAs, which are chemically modified siRNAs with 500 times greater stability in vivo due to their resistance to nuclease activity. These in vivo stable siRNA can provide long-term knockdown of Sp1, Sp3 and Sp4 proteins. The siRNAs are administered by intraperitoneal injection (10-50 μg/mouse). Optimal concentration and dosing frequency for successful knock down of Sp1, Sp3, and Sp4 in the liver and pancreas of mice is determined. These concentrations are then used in the orthotopic model for pancreatic cancer and administered by intraperitoneal injection. The duration of the proposed treatment is 4 weeks, and pancreatic tumors, liver metasasis, histopathology and immunohistochemistry is carried out as described for the non-steroidal anti-inflammatory drugs above. The treatment group size is decreased to five mice due to the limited availability of the siRNAs.

### EXAMPLE 26

### Tolfenamic Acid Inhibited Pancreatic Tumor Growth and Metastasis in an Orthotopic Model

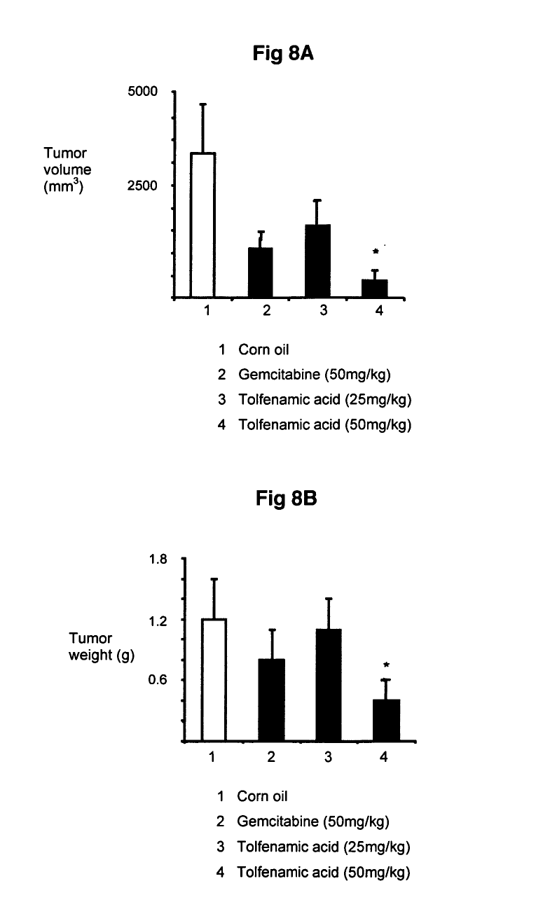

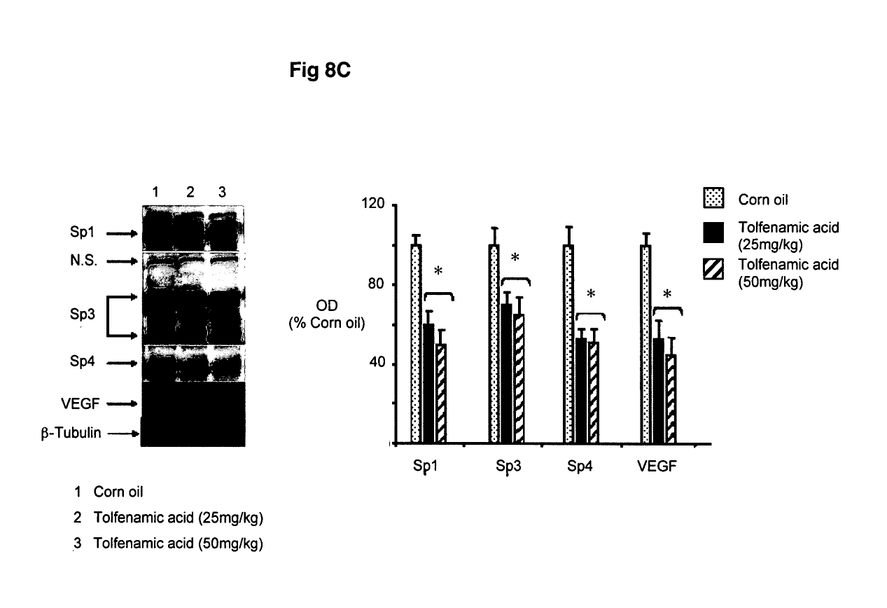

The potential anti-tumorigenic and anti-angiogenic activity of tolfenamic acid against pancreatic tumors was investigated in an orthotopic athymic nude mouse model. L3.6pl cells were used for this study based on their aggressive growth and production of liver metastases (Bruns et al, 1999; Dubois et al, 1998). Tolfenamic acid (25 or 50 mg/kg/d) decreased median tumor weights and volumes (FIGS. 8A and 8B) and these treatments also decreased the percent incidence of liver metastasis (Table 3). Moreover, at this dose level, changes in body/organ weights or organ toxicity were not observed and a summary of median and range of tumor volumes and weights, incidence of liver metastasis, and comparison with the effects of gemcitabine, are presented in Table 3.

TABLE 3
Treatment of orthotopically implanted human pancreatic L3.6p1 cancer cells by
tolfenamic acid and gemcitabine.
Pancreatic Tumor

| | | Tumor | | Tumor | | Incidence | Body | |
|---|---|---|---|---|---|---|---|---|
| | Tumor | Volume (mm³) | | Weight (g) | | Liver | Weight (g) | |
| Group^{a} | Incidence^{b} | Median | Range | Median | Range | metastasis | Median | Range |
| Corn oil | 10/10 | 3587.9 | 985.7-8662.5 | 1.3 | 0.8-1.5 | 5/10 | 24 | 21-24 |
| Gemcitabine | 10/10 | 1186.8 | 606.4-1441.2 | 0.8 | 0.5-1.1 | 5/10 | 24 | 19-29 |
| (50 mg/kg) | | | | | | | | |
| Tolfenamic acid | 10/10 | 1732.8 | 984.7-2076.9 | 1.2 | 0.8-1.3 | 1/10 | 25 | 19-27 |
| (25 mg/kg) | | | | | | | | |
| Tolfenamic acid | 10/10 | 348.8^{c} | 128.3-840.5 | 0.4^{c} | 0.1-0.9 | 1/10 | 24 | 24-27 |
| (50 mg/kg) | | | | | | | | |

^{a}L3.6p1 human pancreatic cancer cells (1 × 10⁶) were injected into the pancreas of nude mice. Seven days later, different groups of mice were treated with bi-weekly intraperitoneal injections of gemcitabine (50 mg/kg), thrice weekly oral tolfenamic acid (25 mg/kg) or (50 mg/kg) or saline (control). All mice were sacrificed on day 35.
^{b}Number of positive mice/number of mice injected.
^{c}p < 0.005 as compared to controls.

The results also indicated that at comparable doses tolfenamic acid was more effective than gemcitabine as a tumor growth inhibitor, whereas the latter compound did not affect the incidence of liver metastasis at the dose used herein. The pancreatic tumors from the orthotopic model were also examined and the levels of Sp1, Sp3, Sp4 and vascular endothelial growth factor proteins in tolfenamic acid versus corn oil control-treated animals were quantitated. Tolfenamic acid treatment significantly decreased expression of Sp1, Sp3, Sp4 and vascular endothelial growth factor in pancreatic tumors (FIG. 8C), which paralleled the same responses observed in pancreatic cancer cells treated with this compound (FIGS. 1 and 4).

Immunostaining for vascular endothelial growth factor in tumor sections from control, gemcitabine (50 mg/kg) and tolfenamic acid (25 and 50 mg/kg) treated animals also showed relatively greater staining in tumors from control and gemcitabine-treated animals, but showed a decreased staining in tumors from mice treated with tolfenamic acid (FIG. 8D). In parallel studies, staining with CD31 to determine microvessel density showed decreased staining in tumors from mice treated with tolfenamic acid compared to tumors from vehicle (corn oil) or gemcitabine-treated animals (FIG. 8E). These results demonstrated that tolfenamic acid exhibited anti-tumorigenic and anti-angiogenic activities in pancreatic cancer cells and tumors in vivo through degradation of Sp proteins which led to decreased vascular endothelial growth factor expression and indicated that the diphenyl/diphenylamine carboxylic acid subclass of non-steroidal anti-inflammatory drugs were promising drugs for treatment of cancers such as pancreatic, breast, prostate, colon, bladder and ovarian cancers.

The following references were cited herein:
Abdelrahim M et al. Cancer Res (2004) 64, 6740-6749
Abdelrahim et al., 2005, Mol Pharmacol 68:317-329.
Abdelrahim M et al. J Biol Chem (2005) 280, 16508-16513
Anderson K E et al. J Natl Cancer Inst (2002) 94, 1168-1171
Baker C H et al. Cancer Res (2002) 62, 1996-2003
Bernier-Villamor V et al. Cell (2002) 108, 345-356
Black A R et al. J Cell Physiol (2001) 188, 143-160
Boolbol S K et al. Cancer Res (1996) 56, 2556-2560
Bouwman P et al. Mol Cell Endocrinol (2002) 195, 27-38
Bruan H et al. Nucleic Acid Res (2001) 29, 4994-5000
Bruns C J et al. Neoplasia (1999) 1, 50-62
Chiefari E et al. BMC Cancer (2002) 2, 35-36
Chiu C H et al. Cancer Res (1997) 57, 4267-4273
Chou T Y et al. Proc Natl Acad Sci USA (1995) 92, 4417-4421
Chu S and Ferro T J. Gene (2005) 348, 1-11
Cramer D W et al. Lancet (1998) 351, 104-107
Dick L R, J Biol Chem (1996) 271, 7273-7276
Dubois et al., 1998, FASEB J 12:1063-1073.
Egan K M et al. J Natl Cancer Inst (1996) 88, 988-993
Gately S et al. Semin Oncol (2004) 31, 2-11.
Giovannucci E et al. N Engl J Med (1995) 333, 609-614
Harris R E et al. Oncol Rep (1999) 6, 71-73
Hart G W. Annu Rev Biochem (1997) 66, 315-335
Hosoi Y et al. Int J Oncol (2004) 25, 461-468
Huang W et al. J Biol Chem (2005) 280, 10047-10054
Jackson S P and Tjian R. Cell (1988) 55, 125-133
Jackson S P et al. Cell (1990), 63, 155-165
Jacobs E J et al. J Natl Cancer Inst (2004) 96, 524-528
Jacobs E J et al. J Natl Cancer Inst (2005a) 97, 975-980
Jacobs E J et al. Cancer Epidemiol Biomarkers Prev (2005b) 14, 261-264
Jacoby R F et al. Cancer Res (2000) 60, 5040-5044
Johnson E S and Gupta A A. Cell (2001) 106, 735-744
Kelly W G et al. J Biol Chem 268, 10416-10424
Khuder S A et al. Br J Cancer (2001) 84, 1188-1192
Labayle D et al. Gastroenterology (1991) 101, 635-639
Lindblad M et al. Cancer Epid Biomarkers Prev (2005) 14, 444-450
Martinez M E et al. Cancer Epidemiol Biomarker Prev (1995) 4, 703-707
Masferrer et al., 2000, Cancer Res 60:1306-1311.
Menezes R J et al. BMC Public Health (2002) 2, 18-19
Norrish A E et al. Int J Cancer (1998) 77, 511-555
Nugent K P et al. Br J Surg (1993) 80, 1618-1619
Omura S J et al. Antibiot (Tokyo) (1991) 44, 113-118
Pelge I I et al. Dig Dis Sci (1996) 41, 1319-1326
Pereir M A et al. Carcinogenesis (1994) 15, 1049-1054
Pichler A et al. Cell (2002) 108, 109-120
Reason A J et al. J Biol Chem (1992) 267, 16911-16921
Reddy S, Cancer Res (2000) 60, 293-297
Rock K et al. Cell (1994) 78, 761-771
Rodriguez C et al. Lancet (1998) 352, 1354-1355
Rodriguez M S et al. J Biol Chem (2001) 276, 12654-12659
Ryu H et al. Proc Natl Acd Sci USA (2003) 100, 4281-4286
Safe S et al. Prog Nucleic Acid Res Mol Biol (2004) 77, 1-36
Sapetschnig A et al. EMBO J (2002) 21, 5206-5215
Sapetschnig A et al. J Biol Chem (2004) 279, 42095-42105
Schernhammer E S et al. J Natl Cancer Inst (2004) 96, 22-28
Sharp C R et al. Br J Cancer (2000) 83, 112-120
Shi Q et al. Cancer Res (2001) 61, 4143-4154
Steinbach G et al. N Engl J Med (2000) 342, 1946-1952
Su K et al. J. Biol Chem (1999) 274, 15194-15202
Suzuki T et al. Genes Cell (2000) 5, 29-41
Taketo M M. J Natl Cancer Inst (1998) 90, 1529-1536
Taketo M M. J Natl Cancer Inst (1998) 90, 1609-1620
Tarnawski A S and Jones M K. J Mol Med (2003) 81, 627-636
Tatham M H et al. J Biol Chem (2001) 276, 35368-35374
Tavani A et al. Ann Oncol (2000) 11, 1171-11173
Thun M et al. Cancer Res (1993) 53, 1322-1327
Thun M et al. J Natl Caner Inst (2002) 94, 252-256
Wang L et al. Clin Cancer Res (2003) 9, 6371-6380
Wei D et al. Cancer Res (2004) 64, 2030-2038
Yao J C et al. Clin Cancer Res (2004) 10, 4109-4117
Zannetti A et al. Cancer Res (2000) 60, 1546-1551
   Any patents or publications mentioned in this specification are indicative of the levels of those skilled in the art to which the invention pertains. Further, these patents and publications are incorporated by reference herein to the same extent as if each individual publication was specifically and individually indicated to be incorporated by reference.

What is claimed is:

1. A method of inducing degradation of one or more Sp transcription factors, comprising:
contacting a cancer cell with a non-steroidal anti-inflammatory drug or a substituted diphenylamine or diphenylamine carboxylic acid derivative, thereby inducing degradation of one or more Sp transcription factors.

2. The method of claim 1, wherein said degradation induces the expression of p27 such that proliferation of the cancer cell is inhibited.

3. The method of claim 1, wherein said degradation decreases the expression of vascular endothelial growth factor or VEGFR1 such that angiogenesis, tumor metastasis, or combination thereof in a tumor comprising said cancer cell is inhibited.

4. The method of claim 1, wherein the non-steroidal anti-inflammatory drug is a diphenyl/diphenylamine carboxylic acid.

5. The method of claim 4, wherein the diphenyl/diphenylamine carboxylic acid is tolfenamic acid, diclofenac sodium, or diflunisal.

6. The method of claim 1, wherein the Sp transcription factor is a Sp1 protein, a Sp3 protein, a Sp4 protein, or a combination thereof.

7. The method of claim 1, wherein the cancer cell is a pancreatic cancer cell, an esophageal cancer cell, a breast cancer cell, a prostate cancer cell, a colon cancer cell, a bladder cancer cell, or an ovarian cancer cell.

8. A method of treating cancer in an individual, comprising:
administering a pharmacologically effective amount of diphenyl/diphenylamine carboxylic acid to the individual, thereby treating the cancer in the individual.

9. The method of claim 8, further comprising:
administering a different chemotherapeutic drug.

10. The method of claim 9, wherein the chemotherapeutic drug is administered concurrently or sequentially with the compound.

11. The method of claim 8, wherein the diphenyl/diphenylamine carboxylic acid inhibits tumor growth, angiogenesis, and/or metastasis in the individual.

12. The method of claim 8, wherein the diphenyl/diphenylamine carboxylic acid is tolfenamic acid, diclofenac sodium, or diflunisal.

13. The method of claim 8, wherein the individual is diagnosed with pancreatic cancer, esophageal cancer, breast cancer, prostate cancer, colon cancer, bladder cancer, or ovarian cancer.

14. A method of treating cancer in an individual, comprising:
administering a pharmacologically effective amount of diphenyl/diphenylamine carboxylic acid; and
a small interfering RNA specific for one or more Sp transcription factors, to the individual, thereby treating the cancer in the individual.

15. The method of claim 14, wherein said cancer treatment inhibits tumor growth, angiogenesis, and/or metastasis in the individual.

16. The method of claim 14, wherein the diphenyl/diphenylamine carboxylic acid is tolfenamic acid, diclofenac sodium, or diflunisal.

17. The method of claim 14, wherein the individual is diagnosed with pancreatic cancer, esophageal cancer, breast cancer, prostate cancer, colon cancer, bladder cancer, or ovarian cancer.

18. The method of claim 14, wherein the Sp transcription factor is a Sp1 protein, a Sp3 protein, a Sp4 protein, or a combination thereof.

19. A method of treating esophageal cancer in an individual, comprising:
administering a pharmacologically effective amount of tolfenamic acid, wherein the tolfenamic acid inhibits proliferation, angiogenesis and/or metastasis of the esophageal cancer, thereby treating the pancreatic cancer in the individual.

20. The method of claim 19, further comprising:
administering a different chemotherapeutic drug.

21. The method of claim 19, wherein the chemotherapeutic drug is administered concurrently or sequentially with the tolfenamic acid.
